(19) United States
(12) Patent Application Publication
(10) Pub. No.: US 2024/0390503 A1
(43) Pub. Date: Nov. 28, 2024

(54) Green Tea-based Nanocomplexes for Eye Disease

(71) Applicants: Agency for Science, Technology and Research, Singapore (SG); Singapore Health Services Pte Ltd, Singapore (SG)

(72) Inventors: Motoichi Kurisawa, Ishikawa (JP); Nunnarpas Yongvongsoontorn, Ishikawa (JP); Joo Eun Chung, Ishikawa (JP); Yasuo Yanagi, Singapore (SG)

(21) Appl. No.: 18/690,862

(22) PCT Filed: Aug. 31, 2022
(86) PCT No.: PCT/SG2022/050631

§ 371 Date: Mar. 11, 2024

(30) Foreign Application Priority Data
Sep. 15, 2021 (SG) 10202110185W

(51) Int. Cl. A61K 47/54; A61K 9/00; A61K 47/61; A61K 47/69; A61P 27/02

(52) U.S. Cl. CPC A61K 47/545; A61K 9/0048; A61K 47/61; A61K 47/6939; A61P 27/02

(57) ABSTRACT

The present invention relates to a composition comprising a self-assembled nanocomplex, wherein the self-assembled nanocomplex comprises one or more active agent physically bound to one or more conjugate, wherein each conjugate comprises one or more flavonoid molecule and a first water-soluble polymer, and the nanocomplex is at least partially encapsulated by a second water-soluble polymer. The present invention also relates to a method of preparing the composition and uses thereof. The present invention also relates to a method of treating an eye disease caused by angiogenesis, comprising administering a composition to a subject in need thereof, wherein the composition comprises a self-assembled nanocomplex, wherein the self-assembled nanocomplex comprises an ophthalmic anti-angiogenesis drug physically bound to one or more conjugate, each conjugate comprising one or more flavonoid molecule and a first water-soluble polymer.

### TECHNICAL FIELD

This invention relates to a composition comprising a self-assembled nanocomplex, its method of preparation and use, in particular in the treatment of eye-disease caused by angiogenesis.

### BACKGROUND ART

Age-related macular degeneration (AMD) is the deterioration of the macula, the small central area of the retina that controls visual acuity. AMD is the leading cause of visual impairment, being third after cataract and glaucoma globally and first in industrialized countries, with a blindness prevalence of 8.7%. There are two stages of AMD: (i) the early non-neovascular AMD which can be diagnosed by accumulation of deteriorating tissue “drusen” seen as yellowish spots in and around the macula, and (ii) the later “wet” neovascular AMD (nAMD), which associates with new blood vessel growth also known as choroidal neovascularization (CNV), originating from choriocapillaries and growing into the retina. These blood vessels are abnormal and lead to leakage of blood and fluid causing permanent damage to the macula. The global prevalence of early, late, and any AMD was found to be 8.01, 0.37, and 8.69%, respectively, with an increasing trend as a consequence of population ageing. The global number of people with AMD is projected to be 196 million in 2020, increasing to 288 million in 2040. Although late nAMD has a lower prevalence than early AMD, it commonly causes sudden and irreversible loss of central vision, accounting for most cases of severe vision loss.

The nAMD treatment is based on neovascularization (angiogenesis) inhibition. The current nAMD standard of care is anti-vascular endothelial growth factor (VEGF) drugs administered via intravitreal (IVT) injections. There are currently five approved anti-VEGF drugs: Ranibizumab (Lucentis®, monoclonal antibody fragment), Pegatinib sodium (Macugen®, pegylated aptamer), Aflibercept (Eylea®, recombinant protein), Brolucizumab (Beovu®, single-chain variable antibody fragment), and Faricimab (Vabysmo™, bispecific antibody targeting VEGF and angiopoietin-2), with one off-label drug, Bevacizumab (Avastin®, monoclonal antibody) which is approved for cancer treatment. The current anti-VEGF injections improve visual acuity somewhat. For example, monthly IVT injection of Ranibizumab (0.5 mg) has been shown to improve the visual acuity of 33.8% of AMD patients. However, these treatments only retard the progression toward complete vision loss, requiring frequently repeated IVT injections for a lifetime. Due to the invasive administration route, these injections cause a high degree of risk, including tissue damage and infection such as retinal detachment, intraocular inflammation, increased intraocular pressure, haemorrhage, eye pains and traumatic cataract, which require secondary treatments and potentially cause permanent vision loss. For example, a two year treatment with Ranibizumab has been shown to increase the incidences of intraocular inflammation and increased intraocular pressure to 18 and 24%, respectively, as compared to untreated patients (8 and 7%, respectively). Moreover, these injections require professional medical practices and incur high treatment costs of approximately US$2,000/dose. It gives rise to not only a concern for patients' quality of life and finances but also to the burden on clinicians with increasing nAMD prevalence as a consequence of an aging population. Therefore, an effective and safe nAMD treatment is urgently required.

To improve the side-effect issues of IVT injection, considerable effort has been devoted to developing nAMD treatment systems with a reduced injection frequency or non-injective routes. For example, Port Delivery System (PDS) with Ranibizumab, is a small permanent non-biodegradable eye implant with a refillable reservoir fixed at the sclera. A self-sealing septum in the centre of the implant flange allows access to the implant reservoir for a drug refill without the need to remove the implant from the eye. The PDS maintained sustained-release of Ranibizumab for more than six months (8.7, 13.0, and 15.0 months in the PDS at 10, 40, and 100 mg/mL, respectively) between implantation of the device and the first required refill. Vision outcomes in the treatment group with a high-dosage of PDS (100 mg/mL) were similar to monthly IVT injections of Ranibizumab. PDS is undergoing phase 3 clinical trials. However, although PDS could reduce the frequency of IVT injection, the device implantation at the sclera still causes side effects including intraocular inflammation and retinal detachment.

Another example of systems aiming for a reduced injection frequency is a formulation of Sunitinib malate (SU, as a pharmaceutical ingredient)-encapsulated microparticles (GB-102). SU is a multi-targeted tyrosine kinase inhibitor that inhibits several receptor tyrosine kinases including VEGF receptor (VEGFR) which has been shown to be involved in the choroidal neovascularization (CNV) development in nAMD. In a phase 1/2a study, IVT injection of the SU-loaded microparticles was well-tolerated with no dose-limiting toxicities, drug-related serious adverse events, or inflammation. A single dose of the SU-loaded microparticles maintained the visual acuity and central retinal thickness of evaluable patients with a reduced overall number of anti-VEGF injections through 6 months in over 80% of treated patients in all dose groups (SU 0.25, 0.5, 1, and 2 mg) The best overall performance was observed with a dose of SU of 1 mg, which was able to control the disease in 7 out of 8 patients for 6 months and in 4 out of 8 patients beyond 8 months. GB-102 is undergoing two phase 2 clinical trials since 2019. However despite the potential for reducing the injection frequency, the concerns for the side effects of IVT injections still remain and increase as long as continuous therapy is required.

To circumvent IVT injections, non-invasive systemic (oral) formulations have been developed. One example is Vorolanib which is a SU analogue and is an oral formulation of a tyrosine kinase inhibitor. In clinical trial phase 1, orally administered Vorolanib has been shown to improve the visual acuity of 24 out of 25 AMD patients who completed the 24 weeks treatment. However, 17% of participants had to discontinue the treatment due to severe systemic adverse effects caused by high oral dosages. Subsequently, the phase 2 study which was completed in 2018 showed a disappointing result that Vorolanib oral treatment did not improve visual acuity as compared to placebo at 52 weeks.

Oral administration has been recognized as one of the most attractive systemic drug delivery routes due to its flexibility in dosage formulations and high patient compliance which comes with ease of administration. However, orally administered drugs are subjected to various environmental issues within the body, including extreme pH variation, enzyme degradation, mucus barrier, and cell penetration through the gastrointestinal tract, which influence drug integrity and absorption as well as limited bioavailability. Moreover, ocular delivery via systemic administration is often hindered due to the blood-ocular barrier that prevents drug penetration into the eye, leading to a drug bioavailability of less than 2%. This low bioavailability forces doses and administration frequency to be high in order to obtain therapeutic concentrations, which may consequently lead to severe systemic toxicity.

In view of the above, there is a need for the development of a composition or method for treating AMD that overcomes or at least ameliorates, one or more of the disadvantages described above.

### SUMMARY

In an aspect, there is provided a composition comprising a self-assembled nanocomplex, wherein the self-assembled nanocomplex comprises one or more active agent physically bound to one or more conjugate, wherein each conjugate comprises one or more flavonoid molecule and a first water-soluble polymer, and the nanocomplex is at least partially encapsulated by a second water-soluble polymer.

Advantageously, a flavonoid-based nanocomplex (NC) is provided in the composition, formed by utilizing the favourable interaction between an active agent and the flavonoid moiety of a flavonoid-polymer conjugate, capable of loading various active agents. Advantageously, the nanocomplex may have a high loading content of the active agent.

Advantageously, the second water-soluble polymer may enable the nanocomplex to have a hydrophilic surface. The presence of the second water-soluble polymer on the nanocomplex surface may promote self-assembly of the one or more active agents with the one or more conjugates to result in greater stability of the nanocomplex as compared to the complex without the layer of the second water-soluble polymer. More advantageously, the presence of the second water-soluble polymer on the nanocomplex surface may allow for a protein to be used as the active agent while the complex without the second water-soluble polymer may not. More advantageously, the additional second water-soluble polymer on the nanocomplex may allow for control of the surface charge of the nanocomplex.

In another aspect, there is also provided a pharmaceutical composition or a pharmaceutical formulation comprising the composition as defined above.

In another aspect, there is provided a method of forming the composition as defined above comprising the steps of:
a) mixing a solution of an active agent and a solution of a flavonoid-first water-soluble polymer conjugate to form a mixture;
b) adding a second water-soluble polymer to the mixture of step (a) to form a nanocomplex;
wherein steps a) and b) may be performed simultaneously or sequentially, and
c) allowing the nanocomplex of step (b) to self-assemble, wherein the nanocomplex comprises the active agent physically bound to the flavonoid-first water-soluble polymer conjugate that is at least partially encapsulated by the second water-soluble polymer, wherein the flavonoid-first water-soluble polymer conjugate comprises one or more flavonoid molecules and a first water-soluble polymer.

Advantageously, the favourable interaction between the active agent and the flavonoid (such as epigallocatechin-3-O-gallate) may result in efficient self-assembly of the nanocomplex and encapsulation of the active agent by the first water-soluble polymer-flavonoid conjugate followed by the second water-soluble polymer.

In another aspect, there is provided the use of the composition as defined above, or the pharmaceutical composition or the pharmaceutical formulation as defined above, in inhibiting endothelial cell proliferation when activated by a proangiogenic growth factor in vitro.

In another aspect, there is provided a composition as defined above, or the pharmaceutical composition or the pharmaceutical formulation as defined above, for use as a medicament.

In another aspect, there is provided a method of treating an eye disease caused by angiogenesis comprising administering to a subject in need thereof a composition as defined above, or the pharmaceutical composition or the pharmaceutical formulation as defined above.

In another aspect, there is provided a composition or the pharmaceutical composition as defined above, or the pharmaceutical formulation as defined above, for use in treating an eye disease caused by angiogenesis.

In another aspect, there is provided the use of a composition as defined above, or the pharmaceutical composition or the pharmaceutical formulation as defined above, in the manufacture of a medicament for treating an eye disease caused by angiogenesis.

In another aspect, there is provided a method of treating an eye disease caused by angiogenesis, comprising administering a composition to a subject in need thereof, wherein the composition comprises a self-assembled nanocomplex, wherein the self-assembled nanocomplex comprises an ophthalmic anti-angiogenesis drug physically bound to one or more conjugate, each conjugate comprising one or more flavonoid molecule and a first water-soluble polymer.

In another aspect, there is provided a composition for use in treating an eye disease caused by angiogenesis, wherein the composition comprises a self-assembled nanocomplex, wherein the self-assembled nanocomplex comprises one or more ophthalmic anti-angiogenesis drug physically bound to one or more conjugate, each conjugate comprising one or more flavonoid molecule and a first water-soluble polymer.

In another aspect, there is provided the use of a composition in the manufacture of a medicament for treating an eye disease caused by angiogenesis, wherein the composition comprises a self-assembled nanocomplex, wherein the self-assembled nanocomplex comprises one or more ophthalmic anti-angiogenesis drug physically bound to one or more conjugate, each conjugate comprising one or more flavonoid molecule and a first water-soluble polymer.

In an example, a green tea-based NC is provided in the composition, formed by utilizing the favourable interaction of drugs and (−)-epigallocatechin-3-O-gallate (EGCG) moiety of a hyaluronic acid (HA)-EGCG conjugate, capable of loading various protein-based and small-molecule VEGF/VEGFR inhibitor drugs. The drug-loaded NCs may be useful in the treatment of neovascular AMD (nAMD), as they may inhibit the in vitro VEGF-induced proliferation of endothelial cells, and may exhibit minimal cytotoxicity under normal growth conditions.

Advantageously, the enhanced inhibitory effect of the drug-loaded NC, as compared to the drug alone, may be attributed to the synergistic effect of the drug and the HA-EGCG carrier. The drug-loaded NCs may show enhanced and sustained anti-angiogenic activity via both topical and intravitreal (IVT) administrations, as compared to free Aflibercept (AF) alone, which is the current standard treatment. The improved efficacy of the NCs may be ascribed to the efficient delivery of drugs to the disease site at the posterior eye segment and carrier-enhanced efficacy. Advantageously, the composition as defined above may be beneficial in both topical administration systems (as a single treatment or combinational treatment with currently existing anti-VEGF therapy) and IVT administration systems, enabling sustained efficacy with a reduced dose of anti-VEGF/VEGFR drugs. Advantageously, this may overcome the problems of the current standard of care for nAMD including injection-related adverse effects, poor patient compliance, burdens on medical practices and high treatment costs.

### Definitions

The following words and terms used herein shall have the meaning indicated:

The word “self-assembled” refers to the process in which a system's components organize into ordered and/or functional structure or patterns as a consequence of specific, local interaction among the local components themselves, without external direction. The word “self-assembly” should be construed accordingly.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF OPTIONAL EMBODIMENTS

### Composition A

There is provided a composition comprising a self-assembled nanocomplex, wherein the self-assembled nanocomplex comprises one or more active agent physically bound to one or more conjugate, wherein each conjugate comprises one or more flavonoid molecule and a first water-soluble polymer, and the nanocomplex is at least partially encapsulated by a second water-soluble polymer.

The flavonoid may be selected from the group consisting of (−)-epicatechin, (+)-epicatechin, (−)-catechin, (+)-catechin, (−)-epicatechin gallate, (+)-epicatechin gallate, epigallocatechin, epigallocatechin gallate, fisetinidol, gallocatechin, gallocatechin gallate, mesquitol, robinetinidol, ellagitannin, gallotannin, oolongtheanin, phlorotannin, tannin, theacitrin, theadibenzotropolone, theaflavin, theanaphthoquinone, thearubigins, theasinensin, quercetin, revastrol, rutin, curcumin isorhamnetin, kaempferol, myricetin, fisetin, hesperitin, naringenin, eriodictyol, genistein, daidzein, cyanidin, delphinidin, malvidin, pelargonidin, peonidin, daidzein, genistein, glycitein, biochanin A, formononetin, apigenin, luteolin, baiocalein, chrysin, and any mixture thereof.

The flavonoid may be a catechin-based flavonoid.

The catechin-based flavonoid may have the following structure:

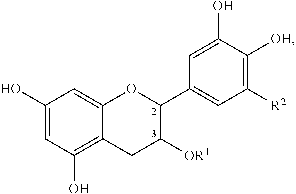

wherein R¹ may be H or galloyl, and
R² may be H or OH.

In the catechin-based flavonoid, there may be two chiral centres at carbons 2 and 3.

The active agent may be in the form of its pharmaceutically acceptable salt or prodrug.

The active agent may be a small molecule, a protein or an oligonucleotide.

The small molecule may be a macromolecule having a molecular weight of less than or equal to 1000 Da. The small molecule may have a molecular weight in the range of 10 Da to about 1000 Da, about 10 Da to about 100 Da or about 100 Da to about 1000 Da.

The active agent may be a therapeutic agent selected from the group consisting of a chemotherapeutic agent, an anti-inflammatory agent, an anti-oxidant agent, an ophthalmic anti-angiogenesis drug and any combination thereof.

The therapeutic agent may be a chemotherapeutic agent selected from the group consisting of alkylating agent, anthracycline, cytoskeletal disruptor, epothilone, histone deacetylase inhibitor, topoisomerase I inhibitor, topoisomerase II inhibitor, kinase inhibitor, monoclonal antibody, antibody-drug conjugate, nucleotide analogue, precursor analogue, peptide antibiotic, platinum-based agent, retinoid, vinca alkaloid, cytokine, anti-metabolite, vinca alkaloid derivative, cytotoxics and any mixture thereof.

The therapeutic agent may be an anti-inflammatory agent selected from the group consisting of aspirin, ibuprofen, naproxen, indomethacin, diclofenac, mefenamic acid, dexamethasone, triamcinolone acetonide, rapamycin, doxycycline, tetracycline, metformin, complement component inhibitors and any mixture thereof.

The therapeutic agent may be an anti-oxidant agent selected from the group consisting of ascorbic acid, vitamin A, vitamin E, melatonin, lipoic acid, metformin and any mixture thereof.

The therapeutic agent may be an ophthalmic anti-angiogenesis drug selected from the group consisting of tyrosine kinase inhibitor, protein, antibody, Sunitinib, Aflibercept, Bevacizumab, Ranibizumab, Pegaptanib sodium, Brolucizumab, Vatalanib, Pazopanib, Sorafenib, Faricimab, squalamine, rapamycin, complement component inhibitors and any mixture thereof.

The therapeutic agent may be the protein Aflibercept (AF). AF is a recombinant fusion protein composed of the second and third extracellular VEGF-binding domains of human VEGFR1 and VEGFR2, respectively, fused to the Fc domain of human IgG1 immunoglobulin. AF may inhibit VEGF-induced angiogenesis by binding to extracellular VEGF. It has been approved as a drug for nAMD treatment via intravitreal (IVT) administration (every 2 months after 3 initial monthly), and is the standard of care for nAMD with a market value of $7.5 billion in 2019.

The therapeutic agent may be the small molecule drug Sunitinib (SU). SU is a small molecule multi-targeted tyrosine kinase inhibitor that may block VEGF signalling by binding to the intracellular ATP-binding site of VEGF. SU requires intracellular delivery to inhibit VEGFR.

The active agent may be present in the composition in the range of from about 0.1 wt % to about 90 wt %, about 0.1 wt % to about 0.2 wt %, about 0.1 wt % to about 0.5 wt %, about 0.1 wt % to about 1 wt %, about 0.1 wt % to about 2 wt %, about 0.1 wt % to about 5 wt %, about 0.1 wt % to about 10 wt %, about 0.1 wt % to about 20 wt %, about 0.1 wt % to about 50 wt %, about 0.2 wt % to about 0.5 wt %, about 0.2 wt % to about 1 wt %, about 0.2 wt % to about 2 wt %, about 0.2 wt % to about 5 wt %, about 0.2 wt % to about 10 wt %, about 0.2 wt % to about 20 wt %, about 0.2 wt % to about 50 wt %, about 0.2 wt % to about 90 wt %, about 0.5 wt % to about 1 wt %, about 0.5 wt % to about 2 wt %, about 0.5 wt % to about 5 wt %, about 0.5 wt % to about 10 wt %, about 0.5 wt % to about 20 wt %, about 0.5 wt % to about 50 wt %, about 0.5 wt % to about 90 wt %, about 1 wt % to about 2 wt %, about 1 wt % to about 5 wt %, about 1 wt % to about 10 wt %, about 1 wt % to about 20 wt %, about 1 wt % to about 50 wt %, about 1 wt % to about 90 wt %, about 2 wt % to about 5 wt %, about 2 wt % to about 10 wt %, about 2 wt % to about 20 wt %, about 2 wt % to about 50 wt %, about 2 wt % to about 90 wt %, about 5 wt % to about 10 wt %, about 5 wt % to about 20 wt %, about 5 wt % to about 50 wt %, about 5 wt % to about 90 wt %, about 10 wt % to about 20 wt %, about 10 wt % to about 50 wt %, about 10 wt % to about 90 wt %, about 20 wt % to about 50 wt %<about 20 wt % to about 90 wt %, or about 50 wt % to about 90 wt % of the total weight of the composition.

The active agent may be loaded via physical interactions (or is “physically bound”) with the flavonoid of the conjugate in the nanocomplex. The physical interaction may be non-covalent. The physical interaction may be selected from the group consisting of ionic bonding, hydrogen bonding, dipole-dipole force, ion-dipole force, ion-induced dipole force, Van der Waals force, hydrophobic interaction, π-π interaction and any mixture thereof.

The first water-soluble polymer may be the same as or may be different from the second water-soluble polymer.

The first water-soluble polymer and the second water-soluble polymer may be independently selected from the group consisting of glycosaminoglycan, polysaccharide, polyacrylamide, poly(N-isopropylacrylamide), poly(oxazoline), polyethylenimine, poly(acrylic acid), polymethacrylate, poly(ethylene glycol), poly(ethylene oxide), poly(vinyl alcohol), poly(vinylpyrrolidinone), polyether, poly(allylamine), polyanhydride, poly(O-amino ester), poly(butylene succinate), polycaprolactone, polycarbonate, polydioxanone, poly(glycerol), polyglycolic acid, poly(3-hydroxypropionic acid), poly(2-hydroxyethyl methacrylate), poly(N-(2-hydroxypropyl)methacrylamide), polylactic acid, poly(lactic-co-glycolic acid), poly(ortho esters), poly(2-oxazoline), poly(sebacic acid), poly(terephthalate-co-phosphate), hyaluronic acid, alginate, amylose, carrageenan, cellulose, cyclodextrin, dextrin, dextran, ficoll, gelatin, gellan gum, guar gum, heparosan, keratin, pectin, polysucrose, pullulan, scleroglucan, starch, xanthan gum, xyloglucan, chitosan and any mixture thereof, or any derivative thereof.

The first and second water-soluble polymer may independently be a mucoadhesive polymer. The mucoadhesive polymer may be selected from the group consisting of a glycosaminoglycan, polysaccharide, poly(hydroxyethyl methylacrylate), poly(ethylene oxide), poly(vinyl pyrrolidone), poly(vinyl alcohol) or poly(acrylic acid) and any mixture thereof, or any derivative thereof. The mucoadhesive polymer may be selected from the group consisting of hyaluronic acid, alginate, amylose, carrageenan, cellulose, cyclodextrin, dextrin, dextran, ficoll, gelatin, gellan gum, guar gum, heparosan, keratin, pectin, polysucrose, pullulan, scleroglucan, starch, xanthan gum, xyloglucan, chitosan and any mixture thereof, or any derivative thereof.

The first and second water-soluble polymer may independently be a biocompatible polymer. The biocompatible polymer may be selected from the group consisting of hyaluronic acid, alginate, amylose, carrageenan, cellulose, cyclodextrin, dextrin, dextran, ficoll, gelatin, gellan gum, guar gum, heparosan, keratin, pectin, polysucrose, pullulan, scleroglucan, starch, xanthan gum, xyloglucan, poly(ethylene glycol), poly(lactide-co-glycolide), polycaprolactone, poly(vinyl pyrrolidone), poly(vinyl alcohol), poly(hydroxyethyl methacrylate), chitosan and any mixture thereof, or any derivative thereof.

The first and second water-soluble polymer may independently be a biodegradable polymer. The biodegradable polymer may be selected from the group consisting of hyaluronic acid, alginate, amylose, carrageenan, cellulose, cyclodextrin, dextrin, dextran, ficoll, gelatin, gellan gum, guar gum, heparosan, keratin, pectin, polysucrose, pullulan, scleroglucan, starch, xanthan gum, xyloglucan, poly(lactide-co-glycolide), polycaprolactone, poly(vinyl pyrrolidone), poly(vinyl alcohol), chitosan and any mixture thereof, or any derivative thereof.

The derivative of the first water-soluble polymer or the derivative of the second water-soluble polymer may comprise the first water-soluble polymer or second water-soluble polymer each independently modified with one or more substituents selected from the group consisting of carboxyl, thiol, sulfonyl, carboxymethyl, phosphoryl, amino, hydroxyl and any combination thereof.

The first water-soluble polymer and the second water-soluble polymer may be non-toxic, biodegradable and/or biocompatible, making them safe to use in biological systems.

The first water-soluble polymer may be hyaluronic acid.

The second water-soluble polymer may be chitosan.

The second water-soluble polymer may at least partially form a shell around a core comprising the one or more active agent physically bound to one or more conjugate, wherein each conjugate comprises one or more flavonoid molecule and a first water-soluble polymer. That is, the nanocomplex may have a core-shell structure. The core may be encapsulated partially or completely.

The shell may be a layer of the second water-soluble polymer which at least partially encapsulates the core comprising the one or more active agent physically bound to one or more conjugate.

The one or more flavonoid molecule may be covalently bound to the first water-soluble polymer.

Advantageously, the one or more flavonoid molecule may be covalently bonded to the first water-soluble polymer to form the conjugate, and the intermolecular interactions between the conjugate (comprising the flavonoid molecules and the first water-soluble polymer) and the active agent may promote drug loading to form the nanocomplex as defined above. As a result, the nanocomplex may not require the use of an additional linker for flavonoid-flavonoid or flavonoid-polymer assembly, which may interfere with the interactions between the active agent and the flavonoid and/or the first water-soluble polymer conjugate.

The conjugate may be termed as a flavonoid-first water-soluble polymer conjugate or a first water-soluble polymer-flavonoid conjugate.

The first water-soluble polymer-flavonoid conjugate may be a hyaluronic acid-epigallocatechin gallate conjugate.

The flavonoid may be epigallocatechin-3-O-gallate (EGCG). EGCG may be one of the major active components of green tea, possessing various health-beneficial effects, including inhibition of VEGF-angiogenic signalling pathway on ocular neovascularization. Advantageously, EGCG may have positive pharmacological benefits, system toxicity, short half-life, low stability and low bioavailability.

The conjugate may comprise epigallocatechin-3-O-gallate and hyaluronic acid.

The conjugate may have the structure depicted by any one of formula (I), (II) or (III):

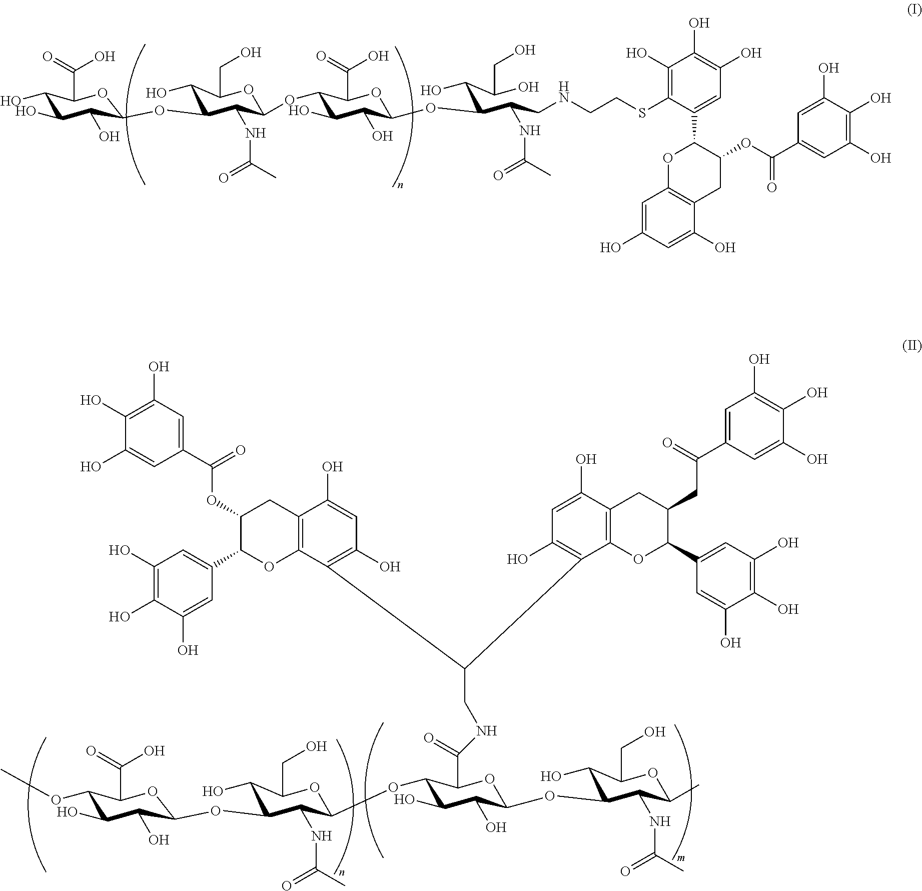

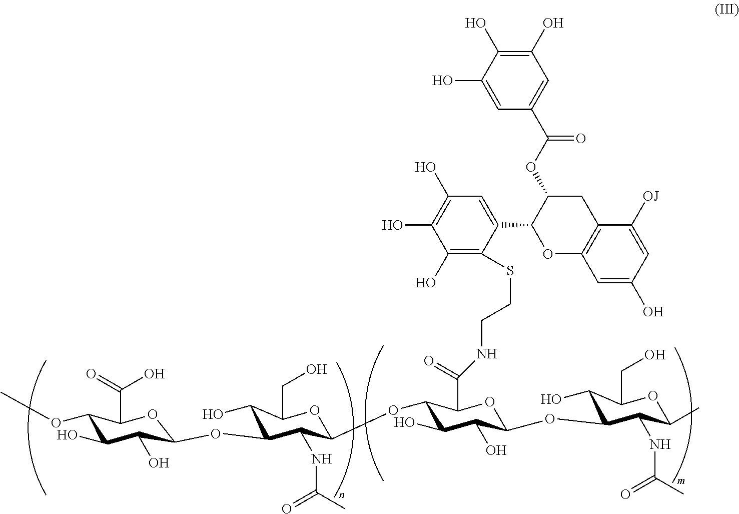

wherein n and m may independently be an integer in the range of 1 to 30,000.
n and m may independently be an integer in the range of 1 to 100, 1 to 200, 1 to 500, 1 to 1000, 1 to 2000, 1 to 5000, 1 to 10,000 or 1 to 30,000, 100 to 200, 100 to 500, 100 to 1000, 100 to 2000, 100 to 5000, 100 to 10,000, 100 to 30,000, 200 to 500, 200 to 1000, 200 to 2000, 200 to 5000, 200 to 10,000, 200 to 30,000, 500 to 1000, 500 to 2000, 500 to 5000, 500 to 10,000, 500 to 30,000, 1000 to 2000, 1000 to 5000, 1000 to 10,000, 1000 to 30,000, 2000 to 5000, 2000 to 10,000, 2000 to 30,000, 5000 to 10,000, 5000 to 30,000, or 10,000 to 30,000.

The conjugate may have a molecular weight in the range of about 1 kDa to about 10,000 kDa, about 1 kDa to about 10 kDa, about 1 kDa to about 100 kDa, about 1 kDa to about 1000 kDa, about 10 kDa to about 100 kDa, about 10 kDa to about 1000 kDa, about 10 kDa to about 10,000 kDa, about 100 kDa to about 1000 kDa, about 100 kDa to about 10,000 kDa or about 1000 kDa to about 10,000 kDa.

Advantageously, the nanocomplex may comprise a hydrophilic surface, favourable size and favourable surface charge.

The nanocomplex may have a hydrodynamic diameter in the range of about 10 nm to about 5000 nm, about 10 nm to about 50 nm, about 10 nm to about 100 nm, about 10 nm to about 500 nm, about 10 nm to about 1000 nm, about 50 nm to about 100 nm, about 50 nm to about 500 nm, about 50 nm to about 1000 nm, about 50 nm to about 5000 nm, about 100 nm to about 500 nm, about 100 nm to about 1000 nm, about 100 nm to about 5000 nm, about 500 nm to about 1000 nm, about 500 nm to about 5000 nm, or about 1000 nm to about 5000 nm.

The nanocomplex may have a polydispersity index in the range of about 0.01 to about 0.50. The nanocomplex may have a polydispersity index of between about 0.01 to about 0.50, about 0.05 to about 0.25, or about 0.09 to about 0.15.

The nanocomplex may have a surface charge in the range of about −60 mV to about 50 mV. The nanocomplex may have a surface charge of about −60 mV to about 50 mV, about −60 mV to about 30 mV, about −60 mV to about 10 mV, about −40 mV to about 50 mV, about −20 mV to about 50 mV, about −60 mV to about −10 mV, about −50 mV to about −20 mV, or about −40 mV to about −30 mV.

The nanocomplex may have a drug loading content in the range of about 0.1% to about 90% by weight, The nanocomplex may have a drug loading content of between about 0.1% to about 90%, about 0.1% to about 70%, about 0.1% to about 50%, about 0.1% to about 30%, about 0.1% to about 10%, about 1% to about 90%, about 10% to about 90%, about 30% to about 90%, about 50% to about 90%, about 70% to about 90%, about 20 wt % to about 70 wt %, about 20 wt % to about 60 wt %, about 20 wt % to about 50 wt %, about 30 wt % to about 70 wt %, about 30 wt % to about 60 wt %, or about 30 wt % to about 50 wt % by weight of the nanocomplex.

The drug loading content may refer to the relative weight of active agent loaded into the nanocomplex in reference to the weight of the nanocomplex.

The nanocomplex may enable effective loading with a wide range of active agents including small molecules, proteins, oligonucleotides and any mixture thereof.

There is also provided a pharmaceutical composition or a pharmaceutical formulation comprising the composition as defined above
The pharmaceutical composition or the pharmaceutical formulation may be a composition or formulation specifically prepared for use in the treatment of a disease or condition.

There is also provided a method of forming the composition as defined above comprising the steps of:
a) mixing a solution of an active agent and a solution of a flavonoid-first water-soluble polymer conjugate to form a mixture;
b) adding a second water-soluble polymer to the mixture of step (a) to form a nanocomplex; wherein steps a) and b) may be performed at the same time or sequentially, and
c) allowing the nanocomplex of step (b) to self-assemble, wherein the nanocomplex comprises the active agent physically bound to the flavonoid-first water-soluble polymer conjugate that is at least partially encapsulated by the second water-soluble polymer, wherein the flavonoid-first water-soluble polymer conjugate comprises one or more flavonoid molecules and a first water-soluble polymer.

The nanocomplex may have a drug loading efficiency in the range of about 20% to about 100% by weight of the active ingredient mixed in step a). The nanocomplex may have a drug loading efficiency of about 20% to about 100%, about 20% to about 80%, about 20% to about 60%, about 20% to about 40%, about 40% to about 100%, about 60% to 100%, about 80% to about 100%, about 60% to about 99%, about 70% to about 98%, about 80% to about 95%, or about 85% to about 95% by weight of the active ingredient mixed in step a).

The drug loading efficiency may refer to the relative weight of the active agent loaded into the nanocomplex in reference to the weight of the active agent mixed in step a) for loading.

There is also provided the use of the composition as defined above, or the pharmaceutical composition or the pharmaceutical formulation as defined above, in inhibiting endothelial cell proliferation when activated by a proangiogenic growth factor in vitro.

The proangiogenic growth factor may be vascular endothelial growth factor, fibroblast growth factor, platelet-derived endothelial growth factor, angiopoietins, hepatocyte growth factor, insulin like growth factors, interleukins and any mixture thereof.

Advantageously, the drug-loaded nanocomplex may display an anti-proliferative effect under proangiogenic growth factor-activated conditions while showing minimal anti-proliferative effect under normal growth conditions. The nanocomplex may exhibit lower IC₅₀ under proangiogenic growth factor activated as compared to conditions without proangiogenic growth factor. Further advantageously, the nanocomplex without the active agent may also display an anti-proliferative effect under proangiogenic growth factor-activated conditions while having minimal effect on cells under normal growth conditions.

There is also provided a composition or the pharmaceutical composition or the pharmaceutical formulation as defined above for use as a medicament.

There is also provided a method of treating an eye disease caused by angiogenesis comprising administering to a subject in need thereof a composition as defined above, or the pharmaceutical composition or the pharmaceutical formulation as defined above.

Advantageously, as compared to a free ophthalmic anti-angiogenesis drug (that is, an ophthalmic anti-angiogenesis drug that is not bound to any delivery vehicle and which is the same ophthalmic anti-angiogenesis drug used in the composition), the composition may result in at least about a sixteenfold higher accumulation of the ophthalmic anti-angiogenesis drug in the retina of the eye than that of free anti-angiogenesis drug. The composition may exhibit at least about a threefold to at least about a sevenfold higher accumulation of drug along the delivery path to the retina, including the cornea, sclera and vitreous humor. Further advantageously, the composition may lead to at least about a fourfold higher accumulation of the nanocomplex in the vitreous humor, suggesting enhanced delivery through not only a trans-sclera route known as the delivery route of nanoparticles but also a corneal route, which eventually facilitate a higher amount of drugs delivered to the retina at the posterior eye segment.

Advantageously, the nanocomplex in the composition may increase bioavailability and delivery of drugs to the posterior eye segment via topical administration. In order to achieve delivery to the disease site at the posterior eye segment, the drugs should firstly be retained at the ocular surface long enough to ensure efficient permeation to the eye tissue. The nanocomplex may increase the precorneal residence time.

Moreover, the nanocomplex in the composition may have favourable properties to pass through the trans-scleral route (conjunctiva→sclera→choroid→retina) and the corneal route (cornea→lens→vitreous humor→retina). Particularly, the trans-scleral route may be favourable for nanocomplexes having a hydrophilic surface, to penetrate through the hydrophilic pores in the sclera and to avoid drug clearance through the conjunctival/choroidal blood vessels and lymphatic drainage, allowing higher permeation to the choroid/retina.

Advantageously, the nanocomplex may be used in topical eye drops for posterior eye disease treatment, enabled by i) the enhanced accumulation at the posterior eye segment through mucoadhesiveness and high affinity of the carrier toward the delivery route and pathologic site, ii) high drug loading, and iii) enhanced efficacy by drug-carrier synergy.

Further advantageously, the nanocomplex may possess a mucoadhesive property, which may allow for prolonged residence time of the nanocomplex at the ocular surface to ensure high drug permeation into the eye, as compared to a free ophthalmic anti-angiogenesis drug.

Further advantageously, the nanocomplex may display optimal physical properties, such as hydrodynamic size, surface charge, loading capacity and stability for favourable transport through the trans-sclera delivery pathway.

Advantageously, the first water-soluble polymer may be hyaluronic acid which may display enhanced affinity to tissues over the delivery route and pathologic area, thereby assisting in the transport of the composition to the angiogenic disease site of the retina. Moreover, a specific binding property of hyaluronic acid to CD44, highly expressed over cornea, conjunctiva, particularly retina and angiogenic vasculature, may preferentially shepherd the loaded drugs to the angiogenic disease site at the retina through the trans-sclera pathway by enhanced affinity to tissues over the delivery route and pathologic area.

Further advantageously, the ophthalmic anti-angiogenesis drug when part of the nanocomplex as defined above may show a synergistic anti-proliferative effect, as compared to a combination of the effects of the individual components (the ophthalmic anti-angiogenesis drug alone and the first water-soluble polymer and flavonoid alone).

Further advantageously, the ophthalmic anti-angiogenesis drug-loaded nanocomplex as defined above may show a synergistic anti-proliferative effect on VEGF-activated endothelial cells, as compared to a combination of the effects of the individual components (the ophthalmic anti-angiogenesis drug alone and first water-soluble polymer-flavonoid conjugate alone).

The nanocomplex may have a combination index of less than 1, less than 0.9, less than 0.8, less than about 0.7, less than about 0.6, less than about 0.5, less than about 0.4, less than about 0.3 or less than about 0.2.

The composition may be in liquid form or solution form for administration to the subject. The composition may be reconstituted from a solid formulation by using a suitable pharmaceutically acceptable buffer that is chosen based on the mode of administration.

As defined herein, “compound” refers to the compounds that are present within the composition of the present disclosure. In accordance with the present disclosure, when used for the treatment of an eye disease caused by angiogenesis, the compounds of the disclosure may be administered alone. Alternatively, the compounds may be administered as a pharmaceutical or veterinarial formulation which comprises at least one compound according to the disclosure. The compound(s) may also be present as suitable salts, including pharmaceutically acceptable salts.

Combinations of active agents, including the compounds of the disclosure, may be synergistic.

By pharmaceutically acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio. Pharmaceutically acceptable salts are well known in the art.

For instance, suitable pharmaceutically acceptable salts of compounds according to the present disclosure may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, oxalic acid, carbonic acid, tartaric acid, or citric acid with the compounds of the disclosure. Suitable pharmaceutically acceptable salts of the compounds of the present disclosure therefore include acid addition salts.

The salts can be prepared in situ during the final isolation and purification of the compounds of the disclosure, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphorsulfonate, citrate, digluconate, cyclopentanepropionate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium potassium, calcium, magnesium, and the like, as well as non-toxic ammonium, quaternary ammonium, and amine cations, including, but not limited to ammonium, tetramethylammonium, tetraethylammonium, methylamine, dimethylamine, trimethylamine, triethylamine, ethylamine, triethanolamine and the like.

Dispersions of the compounds according to the disclosure may also be prepared in glycerol, liquid polyethylene glycols, and mixtures thereof and in oils. Under ordinary conditions of storage and use, pharmaceutical preparations may contain a preservative to prevent the growth of microorganisms.

Pharmaceutical compositions suitable for administration include sterile aqueous solutions (where water soluble) or dispersions and sterile powders for the extemporaneous preparation of sterile injectable solutions or dispersions. Ideally, the composition is stable under the conditions of manufacture and storage and may include a preservative to stabilise the composition against the contaminating action of microorganisms such as bacteria and fungi.

The language “pharmaceutically acceptable carrier” is intended to include solvents, dispersion media, coatings, anti-bacterial and anti-fungal agents, isotonic and absorption delaying agents, and the like. The use of such media and agents for pharmaceutically active substances is well known in the art. Except insofar as any conventional media or agent is incompatible with the compound, use thereof in the therapeutic compositions and methods of treatment and prophylaxis is contemplated. Supplementary active compounds may also be incorporated into the compositions according to the present disclosure. It is especially advantageous to formulate parenteral compositions in dosage unit form for ease of administration and uniformity of dosage. The compound(s) may be formulated for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

Also included in the scope of this disclosure are delayed release formulations.

Compounds of the disclosure may also be administered in the form of a “prodrug”. A prodrug is an inactive form of a compound which is transformed in vivo to the active form. Suitable prodrugs include esters, phosphonate esters etc, of the active form of the compound.

In some examples, the compound may be administered topically or intravitreally. In the case of solutions, the carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of an additive such as lecithin, by the maintenance of the required particle size in the case of dispersion and by the use of surfactants. Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thimerosal, and the like. In many cases, it may be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminium monostearate and gelatin.

Sterile solutions can be prepared by incorporating the analogue in the required amount in an appropriate solvent with one or a combination of ingredients enumerated above, as required, followed by filtered sterilisation. Generally, dispersions are prepared by incorporating the analogue into a sterile vehicle which contains a basic dispersion medium and the required other ingredients from those enumerated above.

Preferably, the pharmaceutical composition may further include a suitable buffer to minimise acid hydrolysis. Suitable buffer-agents are well known to those skilled in the art, but are not limited to, phosphates, citrates, carbonates and mixtures thereof.

The composition may be in the form of a solution where the concentration of the composition in the solution may be in the range of about 0.1 mg/mL to about 100 mg/mL, about 0.1 mg/mL to about 0.2 mg/mL, about 0.1 mg/mL to about 0.3 mg/mL, about 0.1 mg/mL to about 0.5 mg/mL, about 0.1 mg/mL to about 1 mg/mL, about 0.1 mg/mL to about 2 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL, about 0.2 mg/mL to about 0.3 mg/mL, about 0.2 mg/mL to about 0.5 mg/mL, about 0.2 mg/mL to about 1 mg/mL, about 0.2 mg/mL to about 2 mg/mL, about 0.2 mg/mL to about 5 mg/mL, about 0.2 mg/mL to about 10 mg/mL, about 0.2 mg/mL to about 20 mg/mL, about 0.2 mg/mL to about 50 mg/mL, about 0.2 mg/mL to about 100 mg/mL, about 0.5 mg/mL to about 1 mg/mL, about 0.5 mg/mL to about 2 mg/mL, about 0.5 mg/mL to about 5 mg/mL, about 0.5 mg/mL to about 10 mg/mL, about 0.5 mg/mL to about 20 mg/mL, about 0.5 mg/mL to about 50 mg/mL, about 0.5 mg/mL to about 100 mg/mL, about 1 mg/mL to about 2 mg/mL, about 1 mg/mL to about 5 mg/mL, about 1 mg/mL to about 10 mg/mL, about 1 mg/mL to about 20 mg/mL, about 1 mg/mL to about 50 mg/mL, about 1 mg/mL to about 100 mg/mL, about 2 mg/mL to about 5 mg/mL, about 2 mg/mL to about 10 mg/mL, about 2 mg/mL to about 20 mg/mL, about 2 mg/mL to about 50 mg/mL, about 2 mg/mL to about 100 mg/mL, about 5 mg/mL to about 10 mg/mL, about 5 mg/mL to about 20 mg/mL, about 5 mg/mL to about 50 mg/mL, about 5 mg/mL to about 100 mg/mL, about 10 mg/mL to about 20 mg/mL, about 10 mg/mL to about 50 mg/mL, about 10 mg/mL to about 100 mg/mL, about 20 mg/mL to about 50 mg/mL, about 20 mg/mL to about 100 mg/mL, or about 50 mg/mL to about 100 mg/mL.

The composition may be administered once daily, twice daily, thrice daily, once weekly, once fortnightly, once monthly, once every 2 months, once every 3 months, once every 4 months, once every 5 months, once every 6 months, once every 9 months or once every 12 months.

The composition may be administered topically to an eye of the subject.

Non-injective and non-systemic delivery of anti-VEGF/VEGFR drugs may be attained via topical administration. Advantageously, this may significantly improve the safety of the treatment and convenience for patients and clinicians evading the side effects from intravitreal (IVT) injections and minimizing off-target toxicity. Apart from safety, topical administration may have advantages of higher patient compliance due to ease of administration and lower costs. Moreover, it may offer treatment opportunities for patients with limited access to healthcare, especially in rural and underdeveloped areas.

Advantageously, the composition as defined above may be used as a single treatment without having to administer the drug via another administration route such as IVT injections.

The composition may be administered topically at an administration volume of 4 μL, 5 μL, 6 μL, 8 μL, 10 μL, 20 μL, 25 μL, 30 μL, 40 μL, 50 μL, 75 μL, or 100 μL once daily, twice daily or thrice daily.

The composition, when administered topically, may lead to a sustained reduction in retinal lesion development for at least 20 days, at least 25 days, at least 30 days, at least 35 days or the whole period administered.

Advantageously, the composition, when administered topically, may display at least about a 10% reduction in retinal lesion development as compared to when the same ophthalmic anti-angiogenesis drug is administered through intravitreal injection.

Further advantageously, the composition as defined above, when administered topically, may overcome the issues observed in conventional topically administered drugs such as short precorneal residence time and low permeation efficiency of drugs upon topical instillation of eye drop that precludes sufficient delivery to the disease site (choroid/retina) at the posterior eye segment.

The composition may be administered intravitreally to an eye of the subject. The intravitreal administration may be via intravitreal injection.

The composition may be intravitreally administered with an administration volume of 0.5 μL, 1 μL, 2 μL, 5 μL, 10 μL, 15 μL, 20 μL, 25 μL, 30 μL, 40 μL, 50 μL or 100 μL once weekly, once fortnightly, once monthly, once every 2 months, once every 3 months, once every 4 months, once every 5 months, once every 6 months, once every 9 months or once every 12 months.

Advantageously, the composition administered through intravitreal injection may show a sustained anti-angiogenesis effect as compared to a free anti-angiogenesis drug which is administered through intravitreal injection, allowing for reduced injection frequency, even when a lower dose of anti-angiogenesis drug is used in the medicament.

When the composition is administered topically to an eye of the subject, the method may further comprise a step of administering the angiogenesis drug intravitreally to the eye of the subject.

That is, the composition may be administered both topically and intravitreally to an eye of the subject, either simultaneously or sequentially.

Advantageously, such a combined treatment plan may prolong the effect of the anti-angiogenesis drug in inhibiting retinal lesion growth as compared to intravitreal administration alone of the same anti-angiogenesis drug.

The eye disease caused by angiogenesis may be selected from the group consisting of posterior eye disease, age-related macular degeneration, neovascular age-related macular degeneration, diabetic retinopathy, neovascular glaucoma, diabetic macular oedema, retinal vascular occlusion, corneal neovascularization and any combination thereof.

The active agent may be an ophthalmic anti-angiogenesis drug. Advantageously, when the active agent is an ophthalmic anti-angiogenesis drug, the ophthalmic anti-angiogenesis drug when part of the nanocomplex as defined above, may show a synergistic anti-proliferative effect, as compared to a combination of the effects of the individual components (the ophthalmic anti-angiogenesis drug alone and the first water-soluble polymer and flavonoid alone).

Advantageously, when the active agent is an ophthalmic anti-angiogenesis drug, the ophthalmic anti-angiogenesis drug-loaded nanocomplex as defined above may show a synergistic anti-proliferative effect on VEGF-activated endothelial cells, as compared to a combination of the effects of the individual components (the ophthalmic anti-angiogenesis drug alone and first water-soluble polymer-flavonoid conjugate alone).

There is also provided a composition or the pharmaceutical composition as defined above, or the pharmaceutical formulation as defined above, for use in treating an eye disease caused by angiogenesis.

There is also provided the use of a composition or the pharmaceutical composition as defined above, or the pharmaceutical formulation as defined above, in the manufacture of a medicament for treating an eye disease caused by angiogenesis.

### Composition B

There is also provided a method of treating an eye disease caused by angiogenesis, comprising administering a composition to a subject in need thereof, wherein the composition comprises a self-assembled nanocomplex, wherein the self-assembled nanocomplex comprises an ophthalmic anti-angiogenesis drug physically bound to one or more conjugate, each conjugate comprising one or more flavonoid molecule and a first water-soluble polymer.

In the example above, the scope of “eye disease caused by angiogenesis”, “an ophthalmic anti-angiogenesis drug”, “physically-bound”, “conjugate”, “flavonoid” and “first water-soluble polymer” are as defined above for Composition A.

In composition B, the nanocomplex may not be encapsulated by a second water-soluble polymer.

Advantageously, as compared to a free ophthalmic anti-angiogenesis drug (that is, an ophthalmic anti-angiogenesis drug that is not bound to any delivery vehicle and which is the same ophthalmic anti-angiogenesis drug used in the composition), the composition may result in at least about a fourfold higher accumulation of the ophthalmic anti-angiogenesis drug in the retina of the eye than that of free anti-angiogenesis drug. The composition may exhibit at least about a threefold to at least about a ninefold higher accumulation of drug along the delivery path to the retina, including the cornea, sclera and vitreous humor. Further advantageously, the composition may lead to at least about a ninefold higher accumulation of the nanocomplex in the vitreous humor, suggesting enhanced delivery through not only a trans-sclera route known as the delivery route of nanoparticles but also the corneal route, which may eventually facilitate a higher amount of drugs delivered to the retina at the posterior eye segment.

Advantageously, the nanocomplex in the composition may increase bioavailability and delivery of drugs to the posterior eye segment via topical administration. In order to achieve delivery to the disease site at the posterior eye segment, the drugs should firstly be retained at the ocular surface long enough to ensure efficient permeation to the eye tissue. The nanocomplex may increase the precorneal residence time.

Moreover, the nanocomplex in the composition may have favourable properties to pass through the trans-scleral route (conjunctiva→sclera→choroid→retina) and the corneal route (cornea→lens→vitreous humor→retina). Particularly, the trans-scleral route may be favourable for hydrophilic-surfaced nanocomplexes to penetrate through the hydrophilic pores in the sclera and to avoid drug clearance through the conjunctival/choroidal blood vessels and lymphatic drainage, allowing higher permeation to the choroid/retina.

Further advantageously, as compared to a free ophthalmic anti-angiogenesis drug, the composition may allow for treatment of posterior eye disease, enabled by i) the enhanced accumulation at the posterior eye segment through mucoadhesiveness and high affinity of the carrier toward the delivery route and pathologic site, ii) high drug loading, and iii) enhanced efficacy by drug-carrier synergy.

Advantageously, the nanocomplex may possess a mucoadhesive property, which may allow for prolonged residence time of the nanocomplexes at the ocular surface to ensure high drug permeation into the eye, as compared to a free ophthalmic anti-angiogenesis drug.

Further advantageously, as compared to a free ophthalmic anti-angiogenesis drug, the nanocomplex may display optimal physical properties, such as hydrodynamic size, surface charge, loading capacity and stability for favourable transport through the trans-sclera delivery pathway.

Advantageously, the first water-soluble polymer may be hyaluronic acid which may display enhanced affinity to tissues over the delivery route and pathologic area, thereby assisting in the transport of the composition to the angiogenic disease site of the retina. Moreover, a specific binding property of hyaluronic acid to CD44, highly expressed over cornea, conjunctiva, particularly retina and angiogenic vasculature, may preferentially shepherd the loaded drugs to the angiogenic disease site at the retina through the trans-sclera pathway by enhanced affinity to tissues over the delivery route and pathologic area.

Further advantageously, the ophthalmic anti-angiogenesis drug when part of the nanocomplex as defined above may show a synergistic anti-proliferative effect, as compared to a combination of the effects of the individual components (the ophthalmic anti-angiogenesis drug alone and the first water-soluble polymer and flavonoid alone).

Further advantageously, the ophthalmic anti-angiogenesis drug-loaded nanocomplex as defined above may show a synergistic anti-proliferative effect on VEGF-activated endothelial cells, as compared to a combination of the effects of the individual components (the ophthalmic anti-angiogenesis drug alone and first water-soluble polymer-flavonoid conjugate alone).

Further advantageously, the ophthalmic anti-angiogenesis drug when part of the composition as disclosed herein may inhibit endothelial cell proliferation when activated by proangiogenic growth factors in vitro.

Further advantageously, the ophthalmic anti-angiogenesis drug when part of the drug-loaded nanocomplex may display an anti-proliferative effect under proangiogenic growth factor-activated conditions while showing minimal anti-proliferative effect under normal growth conditions. The nanocomplex may exhibit lower IC₅₀ under proangiogenic growth factor activated as compared to conditions without proangiogenic growth factor. Further advantageously, the nanocomplex without the active agent may also display an anti-proliferative effect under proangiogenic growth factor-activated conditions while having minimal effect on cells under normal growth conditions.

Further advantageously, the ophthalmic anti-angiogenesis drug-loaded nanocomplex as defined herein has a combination index of less than about 0.2 to less than about 1.

The composition may be a pharmaceutical composition or a pharmaceutical formulation. The composition may be in liquid form or solution form when ready to administer to a patient. The composition may be reconstituted from a solid formulation by using a suitable pharmaceutically acceptable buffer that is chosen based on the mode of administration.

As defined herein, “compound” refers to the compounds that are present within the composition of the present disclosure. In accordance with the present disclosure, when used for the treatment of an eye disease caused by angiogenesis, compound(s) of the disclosure may be administered alone. Alternatively, the compounds may be administered as a pharmaceutical or veterinarial formulation which comprises at least one compound according to the disclosure. The compound(s) may also be present as suitable salts, including pharmaceutically acceptable salts.

Combinations of active agents, including compounds of the disclosure, may be synergistic.

By pharmaceutically acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio. Pharmaceutically acceptable salts are well known in the art.

For instance, suitable pharmaceutically acceptable salts of compounds according to the present disclosure may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, oxalic acid, carbonic acid, tartaric acid, or citric acid with the compounds of the disclosure. Suitable pharmaceutically acceptable salts of the compounds of the present disclosure therefore include acid addition salts.

The salts can be prepared in situ during the final isolation and purification of the compounds of the disclosure, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphorsulfonate, citrate, digluconate, cyclopentanepropionate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium potassium, calcium, magnesium, and the like, as well as non-toxic ammonium, quaternary ammonium, and amine cations, including, but not limited to ammonium, tetramethylammonium, tetraethylammonium, methylamine, dimethylamine, trimethylamine, triethylamine, ethylamine, triethanolamine and the like.

Dispersions of the compounds according to the disclosure may also be prepared in glycerol, liquid polyethylene glycols, and mixtures thereof and in oils. Under ordinary conditions of storage and use, pharmaceutical preparations may contain a preservative to prevent the growth of microorganisms.

Pharmaceutical compositions suitable for administration include sterile aqueous solutions (where water soluble) or dispersions and sterile powders for the extemporaneous preparation of sterile injectable solutions or dispersions. Ideally, the composition is stable under the conditions of manufacture and storage and may include a preservative to stabilise the composition against the contaminating action of microorganisms such as bacteria and fungi.

The language “pharmaceutically acceptable carrier” is intended to include solvents, dispersion media, coatings, anti-bacterial and anti-fungal agents, isotonic and absorption delaying agents, and the like. The use of such media and agents for pharmaceutically active substances is well known in the art. Except insofar as any conventional media or agent is incompatible with the compound, use thereof in the therapeutic compositions and methods of treatment and prophylaxis is contemplated. Supplementary active compounds may also be incorporated into the compositions according to the present disclosure. It is especially advantageous to formulate parenteral compositions in dosage unit form for ease of administration and uniformity of dosage. The compound(s) may be formulated for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

Also included in the scope of this disclosure are delayed release formulations.

Compounds of the disclosure may also be administered in the form of a “prodrug”. A prodrug is an inactive form of a compound which is transformed in vivo to the active form. Suitable prodrugs include esters, phosphonate esters etc, of the active form of the compound.

In some examples, the compound may be administered topically or intravitreally. In the case of solutions, the carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thimerosal, and the like. In many cases, it may be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminium monostearate and gelatin.

Sterile solutions can be prepared by incorporating the analogue in the required amount in an appropriate solvent with one or a combination of ingredients enumerated above, as required, followed by filtered sterilisation. Generally, dispersions are prepared by incorporating the analogue into a sterile vehicle which contains a basic dispersion medium and the required other ingredients from those enumerated above.

Preferably, the pharmaceutical composition may further include a suitable buffer to minimise acid hydrolysis. Suitable buffer agent agents are well known to those skilled in the art, but are not limited to, phosphates, citrates, carbonates and mixtures thereof.

The composition may be in the form of a solution where the concentration of the composition in the solution may be in the range of about 0.1 mg/mL to about 100 mg/mL, about 0.1 mg/mL to about 0.2 mg/mL, about 0.1 mg/mL to about 0.3 mg/mL, about 0.1 mg/mL to about 0.5 mg/mL, about 0.1 mg/mL to about 1 mg/mL, about 0.1 mg/mL to about 2 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL, about 0.2 mg/mL to about 0.3 mg/mL, about 0.2 mg/mL to about 0.5 mg/mL, about 0.2 mg/mL to about 1 mg/mL, about 0.2 mg/mL to about 2 mg/mL, about 0.2 mg/mL to about 5 mg/mL, about 0.2 mg/mL to about 10 mg/mL, about 0.2 mg/mL to about 20 mg/mL, about 0.2 mg/mL to about 50 mg/mL, about 0.2 mg/mL to about 100 mg/mL, about 0.5 mg/mL to about 1 mg/mL, about 0.5 mg/mL to about 2 mg/mL, about 0.5 mg/mL to about 5 mg/mL, about 0.5 mg/mL to about 10 mg/mL, about 0.5 mg/mL to about 20 mg/mL, about 0.5 mg/mL to about 50 mg/mL, about 0.5 mg/mL to about 100 mg/mL, about 1 mg/mL to about 2 mg/mL, about 1 mg/mL to about 5 mg/mL, about 1 mg/mL to about 10 mg/mL, about 1 mg/mL to about 20 mg/mL, about 1 mg/mL to about 50 mg/mL, about 1 mg/mL to about 100 mg/mL, about 2 mg/mL to about 5 mg/mL, about 2 mg/mL to about 10 mg/mL, about 2 mg/mL to about 20 mg/mL, about 2 mg/mL to about 50 mg/mL, about 2 mg/mL to about 100 mg/mL, about 5 mg/mL to about 10 mg/mL, about 5 mg/mL to about 20 mg/mL, about 5 mg/mL to about 50 mg/mL, about 5 mg/mL to about 100 mg/mL, about 10 mg/mL to about 20 mg/mL, about 10 mg/mL to about 50 mg/mL, about 10 mg/mL to about 100 mg/mL, about 20 mg/mL to about 50 mg/mL, about 20 mg/mL to about 100 mg/mL, or about 50 mg/mL to about 100 mg/mL.

The composition may be administered topically to an eye of the subject.

Non-injective and non-systemic delivery of anti-VEGF/VEGFR drugs may be attained via topical administration. Advantageously, this may significantly improve the safety of the treatment and convenience for patients and clinicians evading the side effects from intravitreal (IVT) injections and minimizing off-target toxicity. Apart from safety, topical administration may have advantages of higher patient compliance due to ease of administration and lower costs. Moreover, it may offer treatment opportunities for patients with limited access to healthcare, especially in rural and underdeveloped areas.

Advantageously, the composition of as defined above may be used as a single treatment without having to administer the drug via another administration route such as IVT injections.

The composition may be administered topically with a volume of 4 μL, 5 μL, 6 μL, 8 μL, 10 μL, 20 μL, 25 μL, 30 μL, 40 μL, 50 μL, 75 μL, or 100 μL once daily, twice daily or thrice daily.

The composition may lead to a sustained reduction in retinal lesion development for at least 20 days, at least 25 days, at least 30 days, at least 35 days, or for the whole period administered. Advantageously, the composition may display at least about a 10% reduction in retinal lesion development as compared to an anti-angiogenesis drug which is administered through intravitreal injection.

Further advantageously, the composition as defined above, when administered topically, may overcome the issues observed in conventional topically administered drugs such as short precorneal residence time and low permeation efficiency of drugs upon topical instillation of eye drop that precludes sufficient delivery to the disease site (choroid/retina) at the posterior eye segment.

The composition may be administered intravitreally to an eye of the subject. The intravitreal administration may be via intravitreal injection.

The composition may be intravitreally administered with a volume of 0.5 μL, 1 μL, 2 μL, 5 μL, 10 μL, 15 μL, 20 μL, 25 μL, 30 μL, 40 μL, 50 μL or 100 μL once weekly, once fortnightly, once monthly, once every 2 months, once every 3 months, once every 4 months, once every 5 months, once every 6 months, once every 9 months or once every 12 months.

Advantageously, the composition administered through intravitreal injection may show a sustained anti-angiogenesis effect as compared to a free anti-angiogenesis drug which is administered through intravitreal injection, allowing for reduced injection frequency and thus greater patient compliance.

When the composition is administered topically to an eye of the subject, the method may further comprise a step of administering the angiogenesis drug intravitreally to the eye of the subject.

That is, the composition may be administered both topically and intravitreally to an eye of the subject, either simultaneously or sequentially.

Advantageously, such a combined treatment plan may prolong the effect of the anti-angiogenesis drug in inhibiting retinal lesion growth as compared to intravitreal administration alone of the same anti-angiogenesis drug, allowing for reduced injection frequency and thus greater patient compliance.

The ophthalmic anti-angiogenesis drug when part of the nanocomplex as defined above, shows a synergistic anti-proliferative effect, as compared to a combination of the effects of the individual components (the ophthalmic anti-angiogenesis drug alone and the first water-soluble polymer and flavonoid alone).

Advantageously, the ophthalmic anti-angiogenesis drug-loaded nanocomplex as defined above may show a synergistic anti-proliferative effect on VEGF-activated endothelial cells, as compared to a combination of the effects of the individual components (the ophthalmic anti-angiogenesis drug alone and first water-soluble polymer-flavonoid conjugate alone).

There is also provided a composition for use in treating an eye disease caused by angiogenesis, wherein the composition comprises a self-assembled nanocomplex, wherein the self-assembled nanocomplex comprises one or more ophthalmic anti-angiogenesis drug physically bound to one or more conjugate, each conjugate comprising one or more flavonoid molecule and a first water-soluble polymer.

There is also provided the use of a composition in the manufacture of a medicament for treating an eye disease caused by angiogenesis, wherein the composition comprises a self-assembled nanocomplex, wherein the self-assembled nanocomplex comprises one or more ophthalmic anti-angiogenesis drug physically bound to one or more conjugate, each conjugate comprising one or more flavonoid molecule and a first water-soluble polymer.

### BRIEF DESCRIPTION OF DRAWINGS

The accompanying drawings illustrate a disclosed embodiment and serve to explain the principles of the disclosed embodiment. It is to be understood, however, that the drawings are designed for purposes of illustration only, and not as a definition of the limits of the invention.

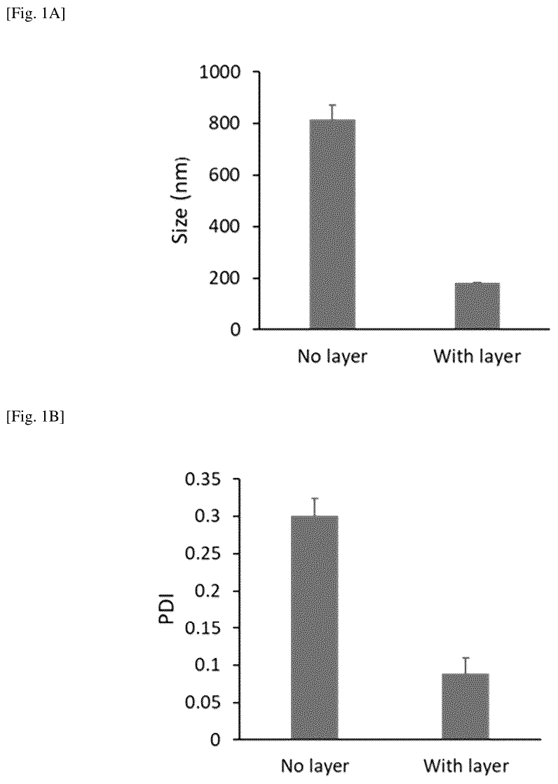

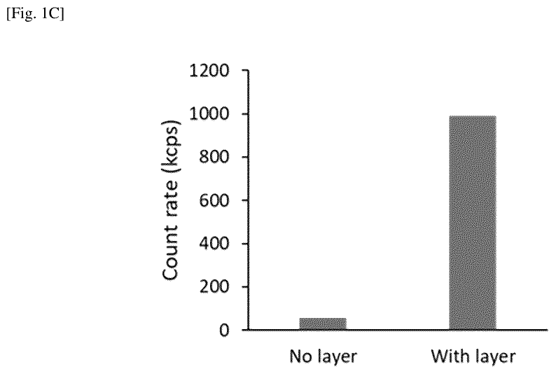

### FIG. 1

FIG. 1A is a graph showing a comparison of the hydrodynamic size of AF-NC with and without the chitosan layer.

FIG. 1B is a graph showing a comparison of the polydispersity index of AF-NC with and without the chitosan layer.

FIG. 1C is a graph showing a comparison of the count rate (i.e. the number of photons detected in dynamic light scattering (DLS) measurements) of AF-NC with and without the chitosan layer. The count rate is related to the concentration (i.e. the number of nanocomplexes) in a sample.

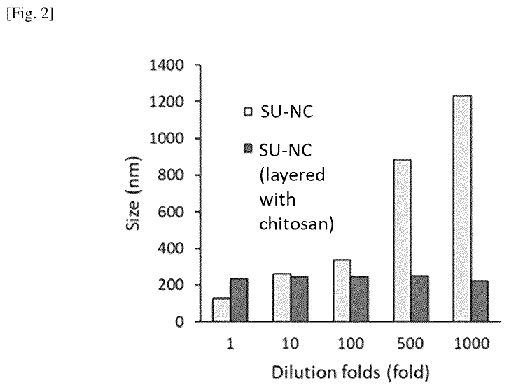

### FIG. 2

FIG. 2 is a graph showing the hydrodynamic size of SU-NC with and without the chitosan layer over increasing folds of dilution.

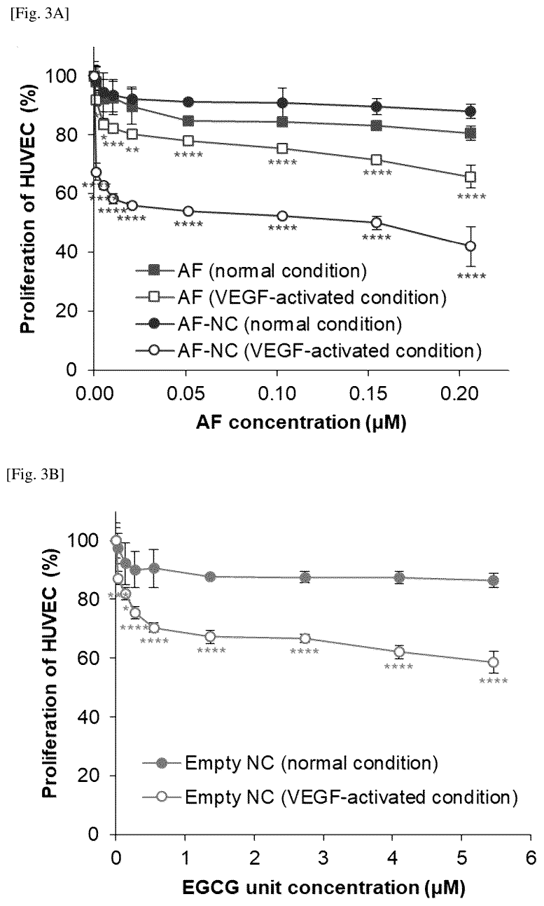

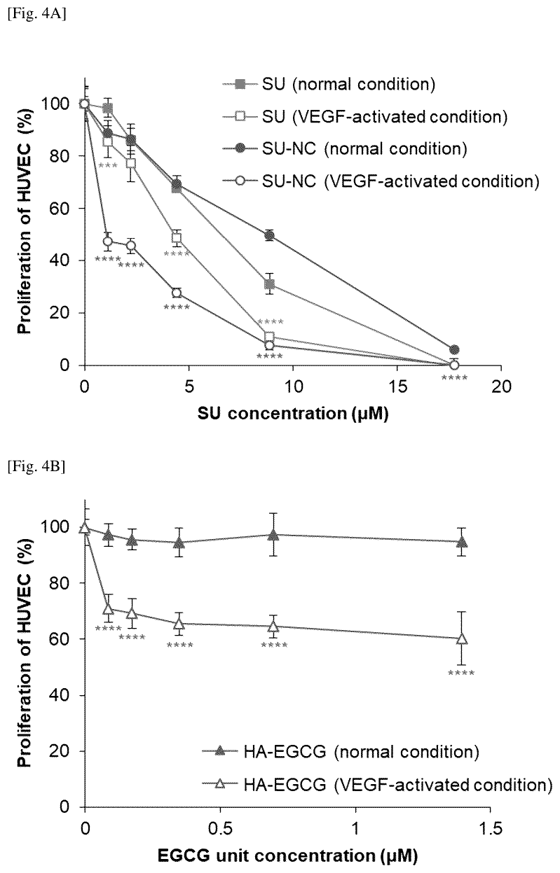

### FIG. 3

FIG. 3 is a set of graphs showing that AF-NC inhibits VEGF-activated proliferation of endothelial cells.

FIG. 3A is a graph showing anti-proliferative effects of AF and AF-NC on HUVEC cultured in normal or VEGF-activated growth condition (n=5, mean±s.d.). *p<0.05; **p<0.01; ***p<0.005; ****p<0.001 (VEGF-activated condition versus normal condition).

FIG. 3B is a graph showing anti-proliferative effects of empty NC (formed with equivalent HA-EGCG/chitosan to AF-NC) on HUVEC cultured in normal or VEGF-activated growth condition (n=5, mean±s.d.). *p<0.05; **p<0.01; ***p<0.005; ****p<0.001 (VEGF-activated condition versus normal condition).

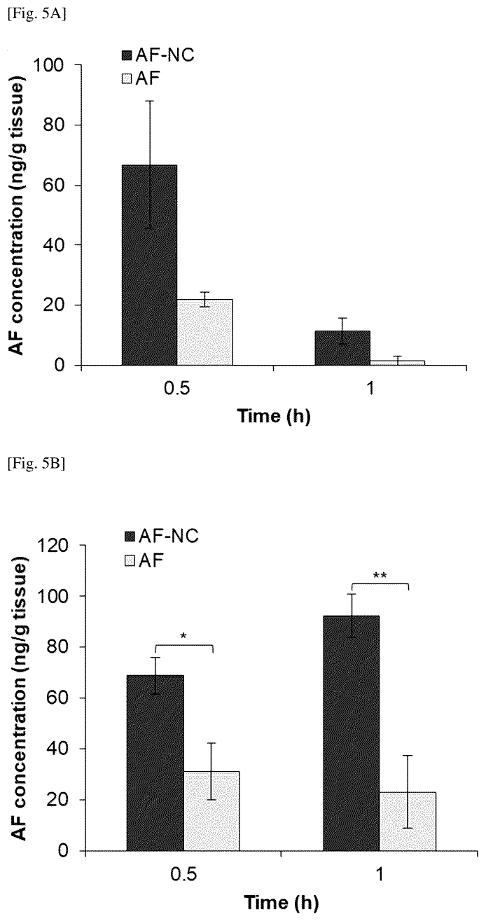

### FIG. 4

FIG. 4 is a set of graphs showing that SU-NC inhibits VEGF-activated proliferation of endothelial cells.

FIG. 4A is a graph showing anti-proliferative effects of SU and SU-NC on HUVEC cultured in normal or VEGF-activated growth condition (n=5, mean±s.d.). ***p<0.005; ****p<0.001 (VEGF-activated condition versus normal condition).

FIG. 4B is a graph showing anti-proliferative effects of HA-EGCG on HUVEC cultured in normal or VEGF-activated growth condition (n=5, mean±s.d.). ***p<0.005; ****p<0.001 (VEGF-activated condition versus normal condition).

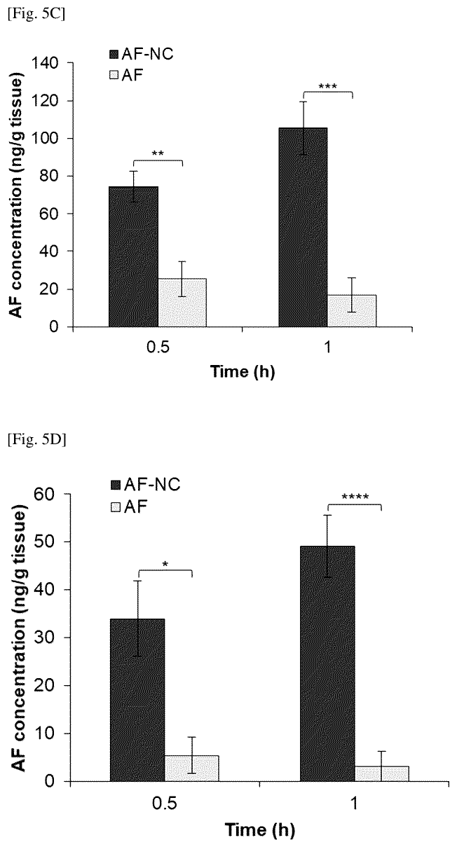

### FIG. 5

FIG. 5 is a set of graphs showing the increased AF accumulation in eye compartments.

FIG. 5A is a graph showing increased AF accumulation in the cornea after topical administration of AF and AF-NC (equivalent AF dose=4 μg) to rat eyes (n=4, mean±s.e.m.). *p<0.05; ***p<0.005; ****p<0.001.

FIG. 5B is a graph showing increased AF accumulation in the vitreous humor after topical administration of AF and AF-NC (equivalent AF dose=4 μg) to rat eyes (n=4, mean±s.e.m.). *p<0.05; ***p<0.005; ****p<0.001.

FIG. 5C is a graph showing increased AF accumulation in the sclera after topical administration of AF and AF-NC (equivalent AF dose=4 μg) to rat eyes (n=4, mean±s.e.m.). *p<0.05; ***p<0.005; ****p<0.001.

FIG. 5D is a graph showing increased AF accumulation in the retina after topical administration of AF and AF-NC (equivalent AF dose=4 μg) to rat eyes (n=4, mean±s.e.m.). *p<0.05; ***p<0.005; ****p<0.001.

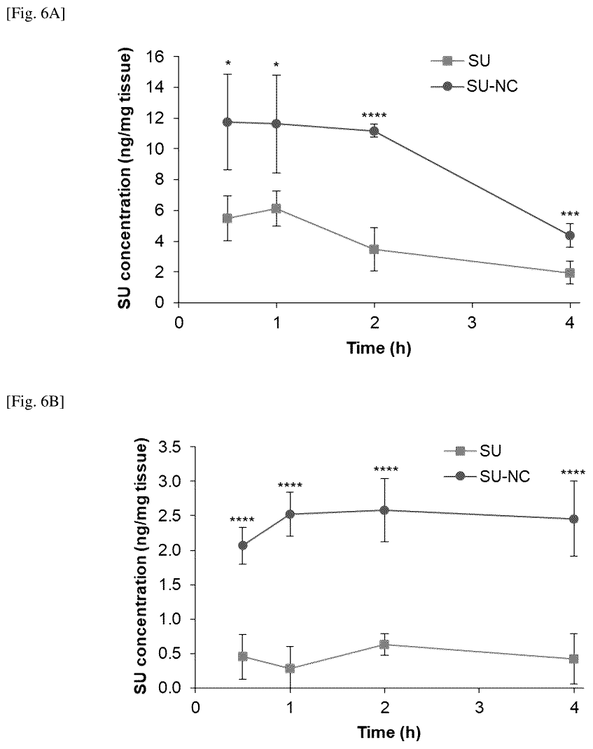

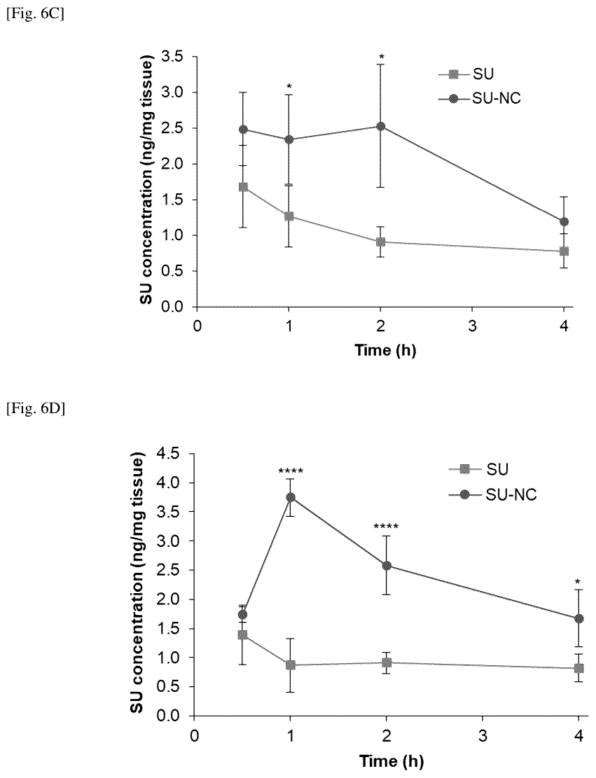

### FIG. 6

FIG. 6 is a set of graphs showing that SU-NC results in increased SU accumulation in eye compartments.

FIG. 6A is a graph showing increased SU accumulation in the cornea after topical administration of SU and SU-NC (equivalent SU dose=10 μg) to rat eyes (n=4, mean±s.d.). *p<0.05; ***p<0.005; ****p<0.001 (SU-NC versus SU).

FIG. 6B is a graph showing increased SU accumulation in the vitreous humor after topical administration of SU and SU-NC (equivalent SU dose=10 μg) to rat eyes (n=4, mean±s.d.). *p<0.05; ***p<0.005; ****p<0.001 (SU-NC versus SU).

FIG. 6C is a graph showing increased SU accumulation in the sclera after topical administration of SU and SU-NC (equivalent SU dose=10 μg) to rat eyes (n=4, mean±s.d.). *p<0.05; ***p<0.005; ****p<0.001 (SU-NC versus SU).

FIG. 6D is a graph showing increased SU accumulation in the retina after topical administration of SU and SU-NC (equivalent SU dose=10 μg) to rat eyes (n=4, mean±s.d.). *p<0.05; ***p<0.005; ****p<0.001 (SU-NC versus SU).

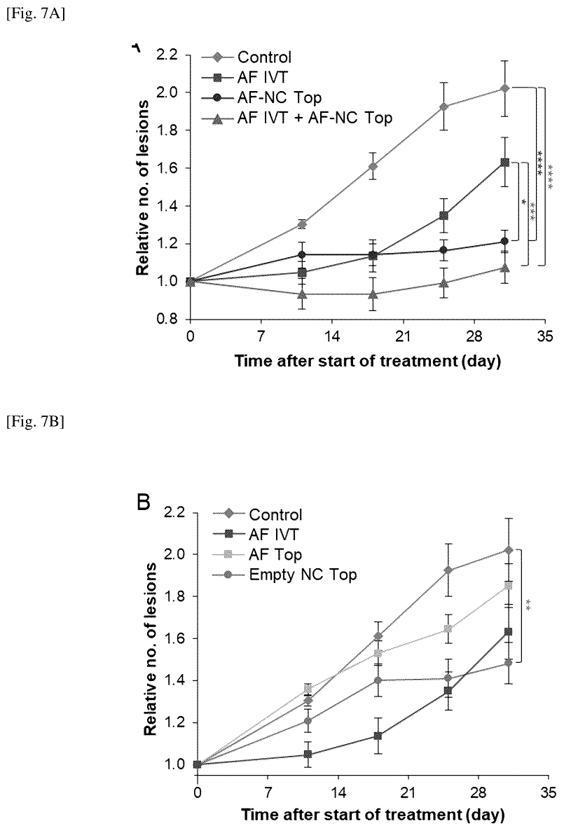

### FIG. 7

FIG. 7 is a set of graphs showing that AF-NC inhibits retinal lesion development in Vldlr^{-/-} mice via topical administration.

FIG. 7A is a graph showing the relative number of lesions of Vldlr^{-/-} mice treated with AF (2.0 μg/0.5 μL, IVT, 1× at day 0), AF-NC (AF 0.2 mg/mL, topical, 10 μL, 3×/day), or combination of AF (2.0 μg/0.5 μL, IVT, 1× at day 0) and AF-NC (AF 0.2 mg/mL, topical, 10 μL, 3×/day) for 31 days (n=4-6, mean±s.e.m.). *p<0.05; **p<0.01; ***p<0.005; ****p<0.001.

FIG. 7B is a graph showing the relative number of lesions of Vldlr^{-/-} mice treated with AF (2.0 μg/0.5 μL, IVT, 1× at day 0), AF (0.2 mg/mL, topical, 10 μL, 3×/day) or empty NC (equivalent HA-EGCG, topical, 10 μL, 3×/day) for 31 days (n=4-6, mean±s.e.m.). *p<0.05; **p<0.01; ***p<0.005; ****p<0.001.

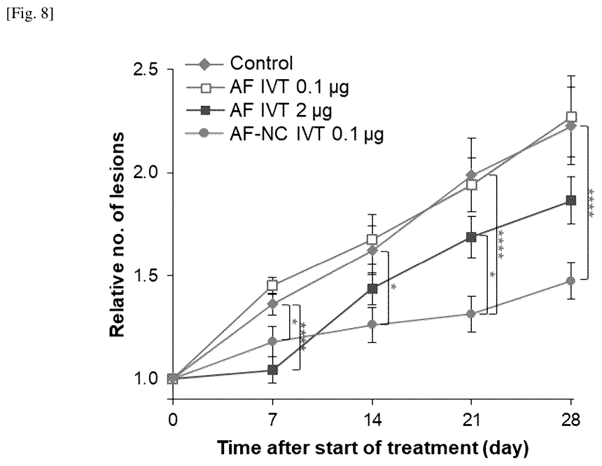

### FIG. 8

FIG. 8 is a graph showing that AF-NC inhibits retinal lesion development in Vldlr^{-/-} mice via IVT injection. The graph indicates the relative number of lesions of Vldlr^{-/-} mice treated with a single IVT injection of AF (0.1 μg/0.5 μL), AF (2.0 μg/0.5 μL) or AF-NC (AF 0.1 μg/0.5 μL) at day 0 (n=5-6, mean±s.e.m.). *p<0.05; ****p<0.001.

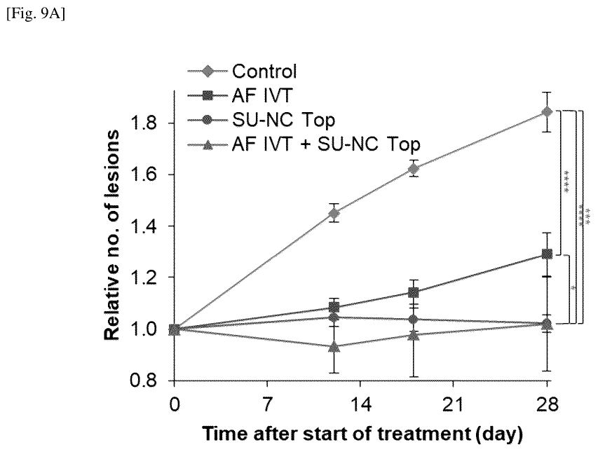

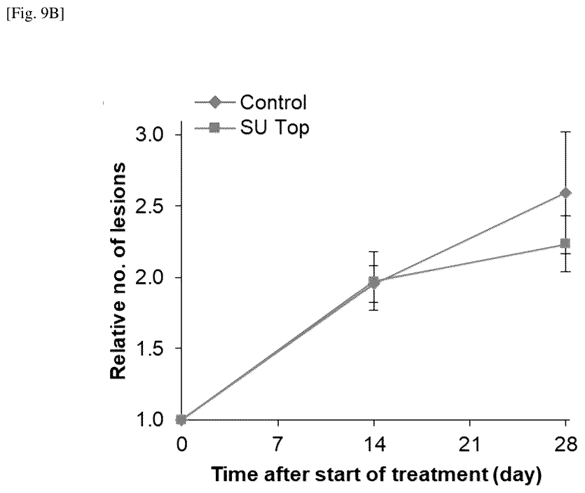

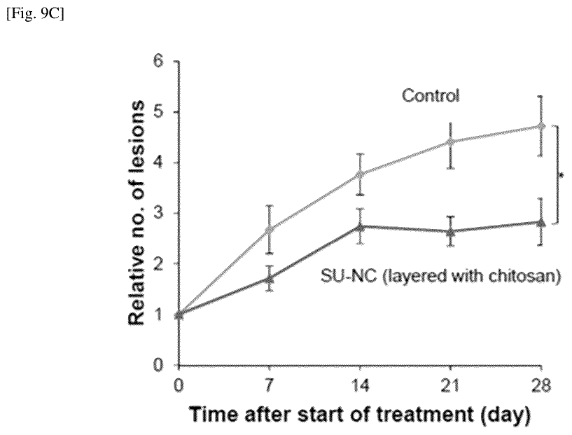

### FIG. 9

FIG. 9 is a set of graphs showing that SU-NC inhibits retinal lesion development in Vldlr^{-/-} mice via topical administration.

FIG. 9A is a graph showing the relative number of lesions of Vldlr^{-/-} mice treated with AF (2.0 μg/0.5 μL, IVT, 1× at day 0), SU-NC (SU 0.17 mg/mL, topical, 10 μL, 3×/day), or combination of AF (2.0 μg/0.5 μL, IVT, 1× at day 0) and SU-NC (SU 0.17 mg/mL, topical, 10 μL, 3×/day) for 28 days (n=5-6, mean±s.e.m.). *p<0.05; ***p<0.005; ****p<0.001.

FIG. 9B is a graph showing the relative number of lesions of Vldlr^{-/-} mice treated with SU (0.5 mg/mL, topical, 10 μL, 3×/day) for 28 days (n=5-6, mean±s.e.m.). *p<0.05; ***p<0.005; ****p<0.001.

FIG. 9C is a graph showing the relative number of lesions of Vldlr^{-/-} mice treated with SU-NC (layered with chitosan) (SU 0.17 mg/mL, topical, 10 μL, 2×/day) for 28 days (n=5-6, mean±s.e.m.). *p<0.05; ***p<0.005; ****p<0.001.

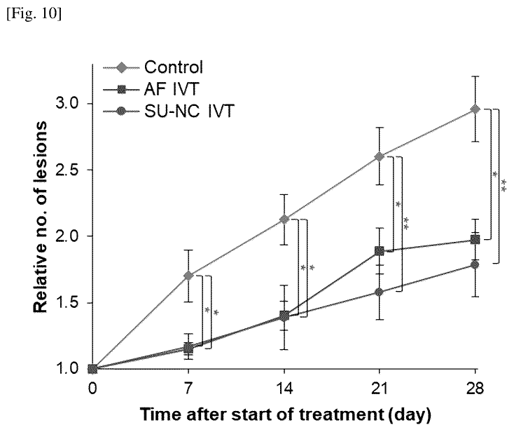

### FIG. 10

FIG. 10 is a graph showing that SU-NC inhibits retinal lesion development in Vldlr^{-/-} mice via IVT injection. The graph shows the relative number of lesions of Vldlr^{-/-} mice treated with a single IVT injection of AF (2.0 μg/0.5 μL) or SU-NC (SU 0.25 μg/0.5 μL) at day 0 (n=5-6, mean±s.e.m.). *p<0.05; **p<0.01.

### EXAMPLES

Non-limiting examples of the invention will be further described in greater detail by reference to specific Examples, which should not be construed as in any way limiting the scope of the invention.

### Materials

Aflibercept (AF) used for the AF-NC formulation was obtained from BOC Sciences (New York City, New York, USA). Clinical grade AF (Eylea® from Bayer (Berlin, Germany)), the current standard treatment for nAMD, was used as a positive control in in vivo studies. Sunitinib malate (SU) was a product of BioVision (Milpitas, California, USA). Amicon Ultra-15 centrifugal filters were purchased from Merck Millipore Corporation (Darmstadt, Germany). All other chemicals were of analytical grade. HUVEC, EBM-2 media and EGM-2 supplements were obtained from Lonza Bioscience Singapore Pte Ltd. (Singapore). Foetal bovine serum (FBS) was obtained from Gibco (Thermo Fisher Scientific Inc, Singapore). Chitosan was obtained from Polysciences (Warrington, Pennsylvania, USA).

### Methods

### Characterization of Nanocomplexes (NCs)

The hydrodynamic diameter and size distribution (polydispersity index) of the NCs were evaluated by dynamic light scattering (DLS) technique using a particle sizer (Brookhaven Instruments, USA) and the surface charge of NCs was determined by Zetasizer Nano ZS (Malvern, UK). The measurements were conducted in triplicate at 25° C.

The amount of SU in SU-NC was determined by measuring the absorbance at 431 nm (UV-VIS U-2810 spectrophotometer, Hitachi, Japan) using the standard curve constructed with various concentrations of SU solutions. The quantity of AF loaded in AF-NC was determined by enzyme-linked immunosorbent assay (ELISA), following the reported procedure with slight modifications. Briefly, 96-well Maxisorp ELISA plates (Nunc, Thermo Fisher Scientific, Waltham, Massachusetts, USA) were coated with 0.2 μg/mL recombinant human VEGF₁₆₅ (i-DNA, Singapore) in phosphate buffer saline (PBS). The plates were blocked with PBS containing 1% bovine serum albumin (BSA) and washed with PBS containing 0.05% TWEEN® 20. Then, the samples were added to the plates, followed by washing with PBS containing 0.05% TWEEN® 20. Subsequently, a horseradish peroxidase (HRP)-conjugated anti-human IgG Fc (Sigma-Aldrich, St. Louis, Missouri, USA) in PBS-BSA was added. After washing the plates, peroxidase activity was assayed by using SureBlue™ tetramethylbenzidine (TMB) microwell peroxidase substrate (KPL) to determine the amount of AF. The absorbance was measured at 405 nm using a microplate reader (Tecan Group Ltd., Mannedorf, Switzerland). The drug loading content and drug loading efficiency were calculated according to the following formulas:

$$Drug loading content ( \% ) = \frac{weight of drug in NC}{total weight of NC} \times 100$$

$$Drug loading efficiency ( \% ) = \frac{weight of drug in NC}{w eight of drug in feed} \times 100$$

### Inhibitory Effects on VEGF-Activated Endothelial Cell Proliferation

Human umbilical vein endothelial cells (HUVECs) were obtained from Lonza Biologics Tuas Pte Ltd (Singapore), and cultured in the endothelial cell basal medium (EBM-2) supplemented with EGM-2 SingleQuots (Lonza Biologics Tuas Pte Ltd, Singapore). HUVECs were seeded in 96-well plates (5×10³ cells/well, n=5) and cultured for one day for cell attachment. After the cells were starved in EBM-2 media containing 0.1% foetal bovine serum (FBS) for 1 day, they were treated with the drug, drug-loaded NC at various drug concentrations (SU: 1.11-17.73 μM, AF: 0.001-0.206 μM), HA-EGCG or empty NC (equivalent HA-EGCG to NC) in two different conditions: in regular media (EBM-2 media supplemented with 2% FBS) or VEGF-supplemented media (EBM-2 media supplemented with 0.1% FBS and 50 ng/mL of recombinant human VEGF₁₆₅ (i-DNA Biotechnology Pte Ltd, Singapore)).

After 3 days, the cell viability was measured by using AlamarBlue® reagent (Life Technologies, USA) according to the manufacturer's protocol. Briefly, the medium was replaced by phenol red-free media containing 10% AlamarBlue® reagent. After incubation for 4 hours at 37° C., fluorescence intensity (λₑₓ=549 nm and λₑₘ=587 nm) was measured by a microplate reader (Tecan Group Ltd., Switzerland). Results were expressed as a percentage of viable cells relative to untreated cells.

The quantitative analysis of the combined effects of drug and HA-EGCG on the VEGF-activated endothelial cell proliferation inhibition of drug-loaded NC was performed using the combination index (CI) based on the Chou-Talalay method which has been widely used for quantifying in vitro and in vivo drug combinations, where CI<1 indicates synergism, CI=1 indicates additive effects, and CI>1 indicates antagonism. The CI values of HA-EGCG and drug combination were calculated with the following equation using the CompuSyn software:

$$CI = \frac{{( D )}_{Drug}}{{( {D}_{x} )}_{Drug}} + \frac{{( D )}_{H A - E G C G}}{{( {D}_{x} )}_{H A - E G C G}}$$

where (D)_{Drug} and (D)_{HA-EGCG} are the doses of drug and HA-EGCG in the drug-loaded NC to achieve x % drug effects, and (Dₓ)_{Drug} and (Dₓ)_{HA-EGCG} are the doses of drug alone and HA-EGCG alone to achieve the same effect.

### Ocular Biodistribution

To analyze in vivo SU distribution in eye, the SU-NC or SU solution (equivalent SU dose=10 μg) was applied topically to eyes of Wistar Hannover rats (InVivos Pte. Ltd., Singapore). At designated time points, the rats were euthanized by carbon dioxide inhalation. The eyes were collected and kept at −80° C. until further processing. The amount of SU in eye compartments were determined by reverse-phase HPLC (Waters 2695 separations module (Waters, USA) equipped with a LaChrom C18-PM column (5 μm, 4.6×250 mm i.d., Hitachi, Japan)), using the standard curves constructed with various concentrations (10-250 ng/mL) of SU in each homogenate eye compartment from untreated rats. UV absorbance was detected at 431 nm. The entire process was performed under minimal light exposure. Homogenates were prepared by homogenizing each eye compartment in phosphate buffer saline (0.1 g of tissue/mL). For extraction, homogenates were mixed with tert-butyl methyl ether (TBME) and shaken (1,300 rpm, 24 hours, 30° C.). After centrifugation, the TBME phase were evaporated under vacuum (45° C.) and reconstituted with the HPLC mobile phase (20 mM ammonium formate (pH 2):acetonitrile=67.5:32.5 (v/v)). The injection volume and flow rate were 20 μL and 0.8 mL/minute, respectively. The amount of SU was determined from the peak integration of SU using Empower 3 software (Waters, USA).

### In Vivo Anti-Angiogenesis Effect on Vldlr^{-/-} Mice

Breeding pairs of very low density lipoprotein receptor gene knock-out (Vldlr^{-/-}) mice (B6; 129S7-Vldlr^{tm1Her}/J) (The Jackson Laboratory, USA) were maintained and bred in standardized conditions (InVivos Pte. Ltd., Singapore). At the age of postnatal day 21 to 28 (P21 to P28), the retinal vasculature was analyzed by fluorescein-angiography. One day later (P22 to P29, day 0), the mice were randomly allocated for AF (2.0 μg/0.5 μL, IVT, 1× at day 0) as a standard treatment, SU (0.5 mg/mL, topical, 10 μL, 3×/day), SU-NC (SU 0.17 mg/mL, topical, 10 μL, 3×/day), AF (0.2 mg/mL, topical, 10 μL, 3×/day), AF-NC (AF 0.2 mg/mL, topical, 10 μL, 3×/day), empty NC (equivalent HA-EGCG/chitosan topical, 10 μL, 3×/day), combination of AF (2.0 μg/0.5 μL, IVT, 1× at day 0) and SU-NC (SU 0.17 mg/mL, topical, 10 μL, 3×/day), or combination of AF (2.0 μg/0.5 μL, IVT, 1× at day 0) and AF-NC (AF 0.2 mg/mL, topical, 10 μL, 3×/day). To study the anti-angiogenesis effect of the NCs via IVT administration, at P22 to 29, the mice were randomly allocated for a single IVT administration of AF (2.0 μg/0.5 μL) as a standard treatment, SU-NC (SU 2.0 μg/0.5 μL), AF-NC (AF 0.1 μg/0.5 μL) or AF at the same concentration to AF-NC (0.1 μg/0.5 μL). The anti-angiogenesis activity was evaluated by the ability to inhibit the development of retinal vascular leakage in terms of relative numbers of lesions.

The care and use of laboratory animals were regulated according to protocols approved by the Institutional Animal Care and Use Committee (IACUC) at the Biological Resource Centre (BRC) in Biopolis, Singapore).

### Fluorescein Angiography

The mice were anesthetized by isoflurane inhalation and the pupils were dilated with 1% tropicamide (Bausch+Lomb, Singapore). Images of the eyes were taken with iVivo® small animal funduscope (OcuScience, USA) after intraperitoneal injection of 0.1 mL of 10% fluorescein sodium (Sigma-Aldrich) in phosphate buffer saline.

### Statistical Analysis

All the data were expressed as mean±standard deviation (s.d.) unless otherwise stated. Statistical analysis was conducted by one-way analysis of variance (ANOVA) using the SigmaStat 3.5 software (Systat Software Inc., USA).

### Example 1: Synthesis

### Synthesis of HA-EGCC

HA-EGCG (I) was synthesized following a procedure previously reported. Briefly, HA was first modified with thiol group at the reducing end. HA (0.5 g) and cystamine dihydrochloride (1.2 g) were dissolved in 30 mL of 0.1 M borate buffer containing 0.4 M NaCl. Sodium cyanoborohydride (628 mg) dissolved in 20 mL of 0.1 M borate buffer was added to the solution. After stirring for 5 days at 37° C., the resulting solution was dialysed (Mw cut-off of 1000 Da) against 0.1 M NaCl solution for two days, 25% ethanol for one day, and deionised water for two days under nitrogen atmosphere. The purified solution was lyophilized to obtain thiol end-modified HA. In the second step, EGCG (440 mg) was mixed with thiol end-modified HA (100 mg) in PBS (70 mL). The mixture was stirred for 4 hours at 25° C. The resulting solution was dialysed (Mw cutoff of 2000 Da) against deionised water under nitrogen atmosphere. The purified solution was lyophilized to obtain HA-EGCG (I).

HA-EGCG (II) was synthesized by a two-step process previously reported. Firstly, to form ethylamine-bridged EGCG dimers, 145 μL of 2,2-diethoxyethylamine (DA) and EGCG (2.3 g) was dissolved in methanesulfonic acid (MSA):tetrahydrofuran (THF) (1:5, v/v, 5 mL). After stirring overnight, the unreacted EGCG was removed by multiple extraction cycles with ethyl acetate. In the second step of conjugating the ethylamine-bridged EGCG dimers to HA, HA (0.25 g), N-hydroxysuccinimide (89 mg), ethylamine-bridged EGCG dimers (0.205 mmol) and 1-ethyl-3-(3-dimethylaminopropyl)-carbodiimide hydrochloride (150 mg) was dissolved in the mixture of MES buffer (19.8 mL) and dimethylformamide (2.5 mL). The reaction mixture was incubated overnight under nitrogen atmosphere. The HA-EGCG (II) was then purified by three cycles of ethanol precipitation and then, the precipitates were re-dissolved in water and dialysed (Mw cutoff of 3500 Da) against deionised water for two days under nitrogen atmosphere before lyophilisation.

HA-EGCG (III) conjugates were synthesized in a two-step procedure previously reported. Firstly, to synthesize thiolated HA, HA (1 g), 4-(4,6-Dimethoxy-1,3,5-triazin-2-yl)-4-methylmorpholinium chloride (1.037 g) and cystamine dihydrochloride (844.5 mg) were mixed in 110 mL of PBS. The resulting mixture was dialysed (Mw cutoff of 3500 Da) against 0.1 M NaCl solution for two days, 25% ethanol for one day and deionised water for two days under nitrogen atmosphere. The purified solution was lyophilised to obtain thiolated HA. In the second step, the thiolated HA (0.5 g) and excess EGCG were mixed in 100 mL of PBS under nitrogen-purged condition. After stirring for 4 hours at 25° C., the mixture was dialysed (Mw cutoff of 3500 Da) against 25% ethanol for one day and deionised water for two days under nitrogen atmosphere.

### Formation of the Drug-Loaded Nanocomplex (NCs)

To formulate AF-NCs, an AF (0.10-0.50 mg/mL) solution and a HA-EGCG (0.10-0.75 mg/mL) solution were mixed. A chitosan solution (0.02-0.30 mg/mL) was added to the solution simultaneously or sequentially. To produce SU-NCs, a SU solution (0.10-0.30 mg/mL) and a HA-EGCG (0.05-0.80 mg/mL) were mixed. For SU-NC with chitosan, a chitosan solution (0.01-0.3 mg/mL) was added to the solution simultaneously or sequentially. The SU-NCs were filtered by centrifugation (5 min, 2,000×g at 25° C.) using Amicon Ultra-15 centrifugal filters (M_{w} cutoff of 100 kDa).

### Example 2: Drug Loaded NCs

AF-loaded NC (AF-NC) was formed by utilizing the EGCG-protein binding property. Upon mixing in an aqueous solution, the HA-EGCG self-assembled with AF through AF-EGCG interaction and was subsequently layered with chitosan on the surface. The AF-NC formation was systematically optimized by varying concentrations and ratios of the compositions in feed. Based on the particle size, surface charge, loading capacity and stability, the AF-NC was optimized for favourable transport through the trans-sclera delivery pathway (hydrodynamic size=204 nm, polydispersity index (PDI)=0.097) (Table 1).

TABLE 1
Characteristics of NCs selected for in vitro inhibitory effects on
VEGF-activated endothelial cell proliferation and in vivo studies.

| | Hydrodynamic | Polydispersity | Surface | Drug loading | Drug loading |
|---|---|---|---|---|---|
| Sample | diameter (nm) | index (PDI) | Charge (mV) | efficiency (%) | content (wt %) |
| SU-NC | 142.3 ± 0.2 | 0.13 ± 0.01 | −38.2 ± 0.3 | 88.4 ± 1.6 | 46.9 ± 0.5 |
| AF-NC | 204.0 ± 3.3 | 0.10 ± 0.01 | −28.9 ± 0.2 | 94.2 ± 1.2 | 34.4 ± 0.3 |

The chitosan introduction yielded the NC with narrow size distribution, while the system without chitosan did not (FIG. 1). It is also worth mentioning that AF loading efficiency of the AF-NC was 94%, demonstrating minimal AF loss during the NC preparation.

SU-loaded NC (SU-NC) was formed by the self-assembly of SU and HA-EGCG through interaction between the SU and EGCG moieties. Notably, SU-NCs could be formulated with and without chitosan. The chitosan introduction enhanced the NC stability retaining its size upon 1000-fold dilution, while the size of the NC without chitosan increased (FIG. 2). The SU-NC formation was systematically optimized by varying concentrations and ratios of the compositions in the feed. Generally, raising the concentration of HA-EGCG led to increased loading efficiency, indicating greater interaction of SU with EGCG at a higher concentration of HA-EGCG. The loading content of the SU-NC initially increases as HA-EGCG/SU (w/w) increases in association with loading efficiency, followed by a decrease in loading content with further increase in HA-EGCG/SU, due to the dominant effect of an increase in HA-EGCG content in the NC. The selected SU-NC without chitosan (hydrodynamic size=142 nm, PDI=0.13, surface charge=−38 mV) was used for in vitro/in vivo investigations (Table 1).

The negative surface charge observed was attributed to the negatively charged HA, demonstrating that the HA covered the NC surface. The SU loading content of the selected SU-NC (46.9%) was markedly higher than those (0.8-5.1 wt %) of reported SU-loaded nanoformulations used in cancer treatment. It was considered that favourable interaction of SU and EGCG moieties along the HA backbone played an important role in the self-assembly of the SU-NC and resulted in efficient encapsulation of SU. Indeed, the non-conjugated HA did not form a particle with SU, showing 3.7% of SU content in the mixture, suggesting that the ionic interaction between positively charged SU and negatively charged HA is not strong enough to form stable NCs.

### Example 3: Inhibitory Effects on VEGF-Activated Endothelial Cell Proliferation

The inhibitory effects of the drug-loaded NCs on the proliferation of human umbilical vein endothelial cells (HUVECs) was investigated in normal or VEGF-induced growth conditions, simulating the nAMD-associated endothelial microenvironment. The AF-NC showed a specific anti-proliferative effect to VEGF-activated conditions with minimal effect (cell viability >87.9%) under normal growth conditions (FIG. 3A), demonstrating a more robust anti-proliferative effect when VEGF signalling pathways were activated. The AF-NC showed much higher anti-proliferative effect (IC₅₀=0.15 μM) than AF (IC₅₀>the maximum concentration tested, 0.21 μM) under VEGF-activated conditions. Noteworthily, empty NC (formed with equivalent HA-EGCG and chitosan) alone showed anti-proliferative effects only when it was VEGF-activated, with minimal cytotoxicity under normal growth conditions (FIG. 3B). The combinatory effects were quantitated by Chou-Talalay method using CompuSyn software. The combination index (CI) at IC₅₀ of the AF-NC was 0.156, illustrating the strong synergism between AF and the empty carrier.

When the inhibitory effect of the SU-NC on the proliferation of HUVECs in normal or VEGF-activated growth conditions was investigated, the SU-NC also showed substantially higher anti-proliferative effects when it was VEGF-activated, compared to normal conditions. SU-NC showed a much higher anti-proliferative effect than SU under VEGF-activated condition, showing a lower IC₅₀ (1.69 μM) than IC₅₀ of SU (4.32 μM) (FIG. 4A). Notably, HA-EGCG alone showed anti-proliferative effects when it was VEGF-activated, but not under normal growth conditions (FIG. 4B). When the combinatory effects of SU and HA-EGCG were quantitated at IC₅₀, the CI value was 0.612, illustrating the strong synergism between SU and the HA-EGCG carrier of the SU-NC. In contrast, under normal growth conditions, SU-NC showed higher IC₅₀ (8.78 μM) than SU (IC₅₀=6.59 μM), indicating a lower cytotoxicity compared to SU for normal growth of endothelial cells.

### Example 4: Delivery to the Posterior Eye Segment Via Topical Administration

The ocular biodistribution of the drug-loaded NC via topical administration was investigated using AF-NC and SU-NC to study the delivery ability to the retina at the posterior eye segment (the pathologic site of nAMD). AF-NC showed 15.6-fold higher AF accumulation in the retina than that of free AF at 1 hour after administration (FIG. 5). SU-NC showed 4.3-fold higher SU accumulation in the retina than that of free SU at 1 hour after administration and maintained the higher accumulation over the whole examined time for 4 hours (FIG. 6), These results demonstrate the excellence of the NC to deliver a high dose of drugs to the retina. The AF-NC and SU-NC exhibited a higher drug accumulation along the path to the retina including the cornea, vitreous humor and sclera, as compared to free drugs. The higher distribution of NCs on the cornea as compared to free drug was considered to be a result of its increased retention time on the cornea through the mucoadhesive/CD44 binding property of HA, which would increase the opportunity for transport and bioavailability in the eye tissues.

Interestingly, NCs showed higher accumulation at not only the sclera but also at the vitreous humor than free drugs, suggesting an enhanced delivery through not only a trans-sclera route known to be the delivery route of nanoparticles but also through a corneal route. This would eventually facilitate a higher amount of drugs delivered to the retina at the posterior eye segment. The improved accumulation of NCs was likely because of the increased bioavailability and efficient delivery of the drug to the posterior eye segment, as a result of taking advantage of the favourably tailored NC formulation.

### Example 5: In Vivo Anti-Angiogenesis Effect on Mice

The in vivo anti-angiogenesis activity of the NCs was investigated on very low density lipoprotein receptor gene knock-out (Vldlr^{-/-}) mice which have been shown to develop choroidal neovascularization (CNV) recapitulating the phenomenon in nAMD. Firstly, the AF-NC was examined for its anti-angiogenesis effect via topical administration. At the age of postnatal day 21 to 28 (P21 to P28), the retinal vasculature was analyzed by fluorescein-angiography. The CNV was determined by the development of retinal vascular leakage in terms of relative numbers of lesions. One day later (P22 to P29, day 0), the mice were randomly allocated for AF (2.0 μg/0.5 μL, IVT, 1× at day 0) as a standard treatment, AF-NC (AF 0.2 mg/mL, topical, 3×/day), AF (0.2 mg/mL, topical, 3×/day), empty NC (equivalent HA-EGCG/chitosan, topical, 3×/day) or combination of AF (2.0 μg/0.5 μL, IVT, 1× at day 0) and AF-NC (AF 0.2 mg/mL, topical, 3×/day).

CNV inhibitory effect of AF (IVT) was observed only in the early stages of treatment and diminished over time. This decline in efficacy is usually observed in patients under the current anti-VEGF treatment, consequently requiring frequent injections. In contrast, AF-NC (topical) significantly retarded the retinal lesion development throughout the experimental period of 31 days (FIG. 7A), while AF (topical) failed to suppress the lesion progression at the same dose (FIG. 7B). These results indicated that the NC successfully delivered AF to the retina via a topical route and achieved high anti-angiogenesis efficacy. In addition, the combination with AF-NC (topical) prevented the deterioration of AF (IVT) efficacy and showed an enhanced and sustained anti-angiogenesis effect as compared to AF (IVT) alone, suggesting the potential of AF-NC (topical) in combinative treatment with current AF (IVT) treatment to prolong the AF efficacy and extend the interval between IVT injections.

Notably, topically administered empty NC also significantly inhibited the progression of retinal lesions (FIG. 7B), indicating the intrinsic anti-angiogenesis effect of HA-EGCG carrier which may contribute to the excellent effect of AF-NC. The anti-angiogenesis efficacy of AF-NC was also examined via IVT administration. A single IVT administration of AF-NC at a 20-fold lower dose (AF 0.1 μg/0.5 μL) showed much greater and sustained inhibitory effects on the progression of retinal lesions compared to AF (2.0 μg/0.5 μL, IVT) which was shown to be effective only in the first 7 days with a diminished effect over time (FIG. 8). This enhanced and prolonged anti-angiogenesis efficacy of AF-NC when administered via IVT injection would facilitate a reduced injection frequency as compared to current anti-VEGF therapy.

Next, the SU-NC was examined for its anti-angiogenesis effect via topical administration. The progression of retinal lesions was significantly inhibited by topically administered SU-NC (SU 0.17 mg/mL) (FIG. 9A), whereas SU (topical) showed no efficacy even at a higher dose of 0.50 mg/mL (FIG. 9B). The SU-NC (topical) showed sustained anti-angiogenesis efficacy, resulting in a significantly higher CNV inhibitory effect than AF (IVT) at 28 days after the start of treatment. When administered in combination, SU-NC (topical) prevented the deterioration of AF (IVT) efficacy and showed enhanced and sustained anti-angiogenesis effect as compared to AF (IVT) alone, suggesting the potential of SU-NC (topical) as a combinational treatment with current AF (IVT) treatment to prolong the AF efficacy and decrease the IVT injection frequency. The progression of retinal legions was significantly inhibited by topically administered SU-NC layered with chitosan (SU 0.17 mg/mL) (FIG. 9C). When SU-NC was administered via IVT injection, a single IVT administration of SU-NC (SU 0.25 μg/0.5 μL) achieved sustained anti-angiogenesis efficacy as compared to AF (2.0 μg/0.5 μL, IVT) (FIG. 10), suggesting the high potential for reduced injection frequency.

### INDUSTRIAL APPLICABILITY

The composition as defined above may be useful in drug delivery, specifically in delivering an ophthalmic anti-angiogenesis drug to treat eye disease caused by angiogenesis, such as inflammatory eye disease, dry eye, cataracts and eye cancer.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A composition comprising a self-assembled nanocomplex, wherein the self-assembled nanocomplex comprises one or more active agent physically bound to one or more conjugate, wherein each conjugate comprises one or more flavonoid molecule and a first water-soluble polymer, and the nanocomplex is at least partially encapsulated by a second water-soluble polymer.

2. The composition according to claim 1, wherein the flavonoid is selected from the group consisting of (−)-epicatechin, (+)-epicatechin, (−)-catechin, (+)-catechin, (−)-epicatechin gallate, (+)-epicatechin gallate, epigallocatechin, epigallocatechin gallate, fisetinidol, gallocatechin, gallocatechin gallate, mesquitol, robinetinidol, ellagitannin, gallotannin, oolongtheanin, phlorotannin, tannin, theacitrin, theadibenzotropolone, theaflavin, theanaphthoquinone, thearubigins, theasinensin, quercetin, revastrol, rutin, curcumin isorhamnetin, kaempferol, myricetin, fisetin, hesperitin, naringenin, eriodictyol, genistein, daidzein, cyanidin, delphinidin, malvidin, pelargonidin, peonidin, daidzein, genistein, glycitein, biochanin A, formononetin, apigenin, luteolin, baiocalein, chrysin, and any mixture thereof.

3. (canceled)

4. The composition according to claim 1, wherein the active agent is a small molecule, a protein or an oligonucleotide.

5. The composition according to claim 1, wherein the active agent is a therapeutic agent selected from the group consisting of a chemotherapeutic agent, an anti-inflammatory agent, an anti-oxidant agent, an ophthalmic anti-angiogenesis drug, and any combination thereof, or wherein the active agent is present in the range of 0.1 wt % to 90 wt % of the total weight of the composition.

6. The composition according to claim 1, wherein the first water-soluble polymer is the same as or is different from the second water-soluble polymer.

7. The composition according to claim 6, wherein the first water-soluble polymer and the second water-soluble polymer are independently selected from the group consisting of glycosaminoglycan, polysaccharide, polyacrylamide, poly(N-isopropylacrylamide), poly(oxazoline), polyethylenimine, poly(acrylic acid), polymethacrylate, poly(ethylene glycol), poly(ethylene oxide), poly(vinyl alcohol), poly(vinylpyrrolidinone), polyether, poly(allylamine), polyanhydride, poly(β-amino ester), poly(butylene succinate), polycaprolactone, polycarbonate, polydioxanone, poly(glycerol), polyglycolic acid, poly(3-hydroxypropionic acid), poly(2-hydroxyethyl methacrylate), poly(N-(2-hydroxypropyl)methacrylamide), polylactic acid, poly(lactic-co-glycolic acid), poly(ortho esters), poly(2-oxazoline), poly(sebacic acid), poly(terephthalate-co-phosphate), hyaluronic acid, alginate, amylose, carrageenan, cellulose, cyclodextrin, dextrin, dextran, ficoll, gelatin, gellan gum, guar gum, heparosan, keratin, pectin, polysucrose, pullulan, scleroglucan, starch, xanthan gum, xyloglucan, chitosan, and any mixture thereof, or any derivative thereof.

8. The composition according to claim 1, wherein the one or more flavonoid molecule is covalently bound to the first water-soluble polymer.

9. The composition according to claim 1, wherein the conjugate has the structure depicted by any one of formula (I), (II), or (III): 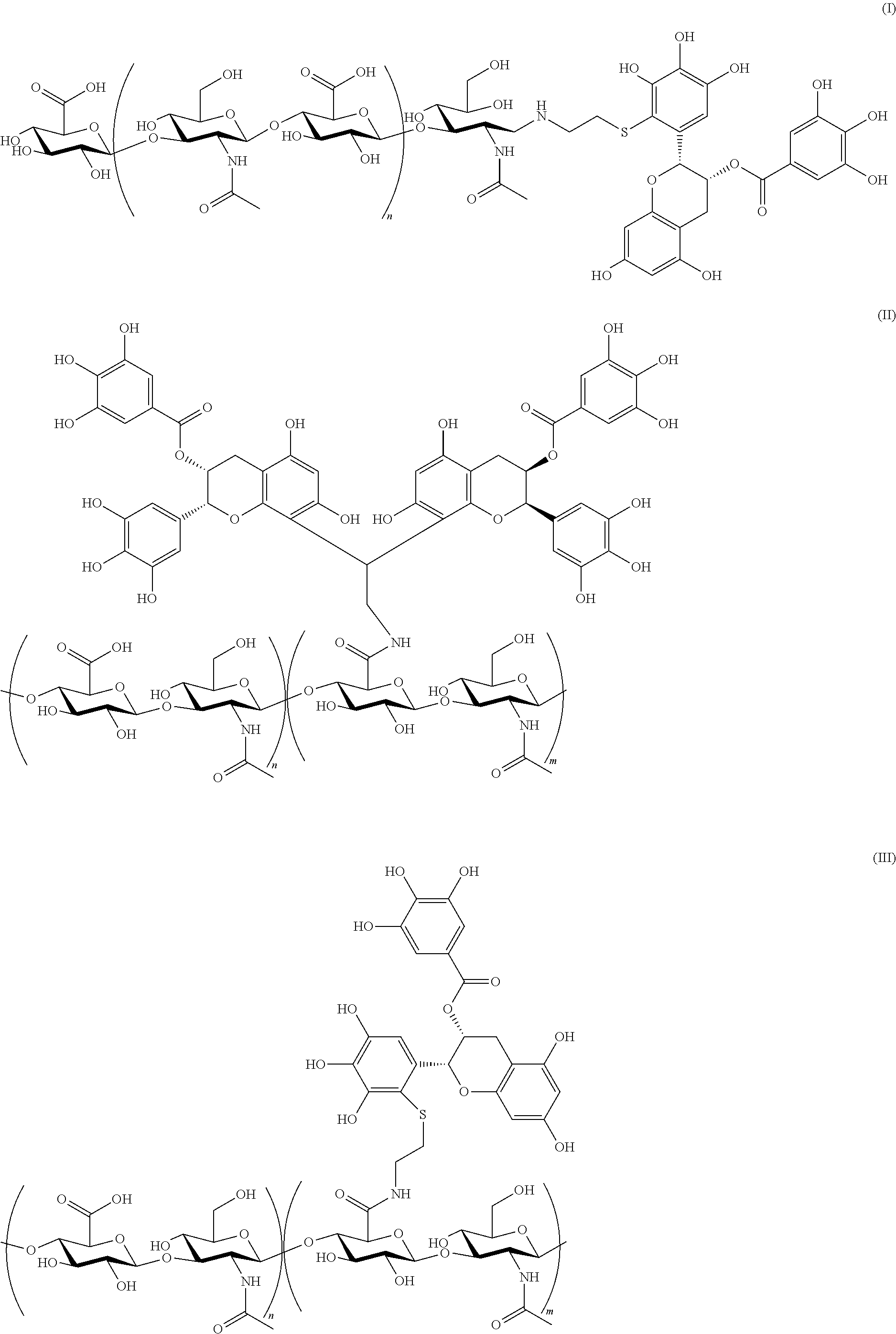
wherein n and m are independently an integer in the range of 1 to 30,000.

10. The composition according to claim 1, wherein the conjugate has a molecular weight in the range of 1 kDa to 10,000 kDa.

11. The composition according to claim 1, wherein the nanocomplex has a hydrodynamic diameter in the range of 10 nm to 5000 nm, a polydispersity index in the range of 0.01 to 0.50, a surface charge in the range of −60 mV to 50 mV, or a drug loading content in the range of 0.1% to 90% by weight of the nanocomplex.

12. A pharmaceutical composition or a pharmaceutical formulation comprising the composition as defined in claim 1.

13. A method of forming the composition according to claim 1 comprising the steps of:
a) mixing a solution of an active agent and a solution of a flavonoid-first water-soluble polymer conjugate to form a mixture;
b) adding a second water-soluble polymer to the mixture of step (a) to form a nanocomplex;
wherein steps a) and b) may be performed simultaneously or sequentially, and
c) allowing the nanocomplex of step (b) to self-assemble, wherein the nanocomplex comprises the active agent physically bound to the flavonoid-first water-soluble polymer conjugate that is at least partially encapsulated by the second water-soluble polymer, wherein the flavonoid-first water-soluble polymer conjugate comprises one or more flavonoid molecules and a first water-soluble polymer.

14. A method of inhibiting endothelial cell proliferation when activated by a proangiogenic growth factor in vitro comprising administering the composition according to claim 1, or a pharmaceutical composition or a pharmaceutical formulation comprising the composition as defined in claim 1.

15. (canceled)

16. A method of treating an eye disease caused by angiogenesis comprising administering a composition according to claim 1, or a pharmaceutical composition or a pharmaceutical formulation comprising the composition as defined in claim 1 to a subject in need thereof.

17. The method according to claim 16, wherein the composition is administered topically and/or intravitreally to an eye of the subject.

18. (canceled)

19. The method of claim 17, further comprising administering an angiogenesis drug intravitreally to the eye of the subject.

20. The method according to claim 16, wherein the eye disease is selected from the group consisting of posterior eye disease, age-related macular degeneration, neovascular age-related macular degeneration, diabetic retinopathy, neovascular glaucoma, diabetic macular oedema, retinal vascular occlusion, and corneal neovascularization.

21. The method according to claim 16, wherein the composition is in liquid form or solution form.

22. The method according to claim 1, wherein the composition is administered once daily, twice daily, thrice daily, once weekly, once fortnightly, once monthly, once every 2 months, once every 3 months, once every 4 months, once every 5 months, once every 6 months, once every 9 months, or once every 12 months.

23.-24. (canceled)

25. A method of treating an eye disease caused by angiogenesis, comprising administering a composition to a subject in need thereof, wherein the composition comprises a self-assembled nanocomplex, wherein the self-assembled nanocomplex comprises an ophthalmic anti-angiogenesis drug physically bound to one or more conjugate, each conjugate comprising one or more flavonoid molecule and a first water-soluble polymer.

26.-27. (canceled)
